(19) United States
(12) Patent Application Publication
(10) Pub. No.: US 2026/0234626 A1
(43) Pub. Date: Aug. 13, 2026

(54) DOWNREGULATION OF RHODOPSIN AS A THERAPEUTIC STRATEGY FOR PERIPHERIN-2-ASSOCIATED INHERITED RETINAL DISORDERS

(71) Applicant: University of Houston System, Houston, TX (US)

(72) Inventors: Muayyad Al-Ubaidi, Houston, TX (US); Muna Naash, Houston, TX (US); Christian Rutan Woods, Houston, TX (US)

(73) Assignee: University of Houston System, Houston, TX (US)

(21) Appl. No.: 19/472,833

(22) PCT Filed: Apr. 2, 2024
(86) PCT No.: PCT/US24/22605

§ 371 Date: Oct. 6, 2025

Related U.S. Application Data
(60) Provisional application No. 63/457,947, filed on Apr. 7, 2023.

(51) Int. Cl. C12N 15/113; A61K 31/712; A61K 31/7125; A61P 27/02

(52) U.S. Cl. CPC C12N 15/113; A61K 31/712; A61K 31/7125; A61P 27/02; C12N 2310/11; C12N 2310/315; C12N 2310/321; C12N 2310/346; C12N 2320/30

(57) ABSTRACT

Reducing RHO levels is an effective therapeutic strategy to ameliorate the disease phenotype in patients with PRPH2-associated inherited retinal disorders. Preferred embodiments for the downregulation of rhodopsin utilize antisense oligonucleotides (ASO) that lead to producing less rhodopsin such as by degradation of rhodopsin mRNA.

### BACKGROUND

This application claims priority to U.S. Provisional Patent Application Ser. No. 63/457,947, entitled “Downregulation of Rhodopsin as a Therapeutic Strategy for Peripherin-2-Associated Inherited Retinal Disorders,” filed Apr. 7, 2023, the entire contents of which are incorporated by reference herein.

Inherited retinal diseases (IRDs) encompass a wide range of heterogeneous disorders resulting in the degeneration of photoreceptors and progressive visual impairment worldwide. Advancements in genetic engineering have shed light on their etiology, leading to the development of the first FDA-approved IRD gene therapy and various non-Prph2 related clinical clinical trials.

Of the many genes associated with IRDs, pathogenic variants in Peripherin-2 (PRPH2) make up some of the most prevalent disease-causing pathogenic variants with over 200 already identified. Pathogenic variants in PRPH2 lead to the formation of aberrantly elongated rod outer segments discs and lead to retinal degeneration. The underlying mechanisms leading to the degeneration remain poorly understood. PRPH2 is a photoreceptor-specific tetraspanin located in the photoreceptor outer segment (OS) disc rim region and forms homo-tetramers as well as hetero-tetramers with its homologue, rod outer segment membrane protein 1 (ROM1). These tetramers subsequently assemble into octamers and higher order oligomers, which play a crucial role in OS disc rim formation. Minimum threshold PRPH2 protein levels (~80% of wild type) are known to be essential for OS disc morphogenesis and maintenance. Therefore, mice heterozygous for the Prph2 null allele (Prph2^{+/-}) exhibit highly abnormal OSs, while homozygotes (Prph2^{-/-}) fail to develop OSs.

To date, there are no approved treatments or ongoing clinical trials specifically for PRPH2 associated diseases. While many studies have provided insights into the correlation between PRPH2 genotypes and the resulting phenotypes in animal models, establishing these associations in human patients is challenging. This difficulty arises from the substantial degree of interfamilial and intrafamilial phenotypic variability observed among individuals carrying identical PRPH2 pathogenic variants and, the complexity is further compounded by the occurrence of digenic inheritance involving its binding partner ROM1. PRPH2 pathogenic variants can exert their effects through loss-of-function, dominant-negative, and/or gain-of-function mechanisms, making traditional gene supplementation therapies inapplicable for all pathogenic variants. Supplementation alone cannot effectively treat dominant-negative and gain-of-function pathogenic variants, while combining gene knockdown with supplementation could be a potential solution. However, the large number of unique pathogenic variants and their low occurrence renders this approach economically unfeasible. These complex pathogenic mechanisms along with the vast array of different disease-causing pathogenic variants makes the identification of a ubiquitous target for the treatment of PRPH2-associated diseases highly desirable.

The ratio of PRPH2 to the rod-specific protein rhodopsin (RHO) plays a crucial role in morphogenesis of rod OS discs. RHO is a light-sensitive protein located in the disc lamellae and is the most abundant retinal protein. It is highly regulated due to its essential role in phototransduction and visual function. Murine models with an increased RHO/PRPH2 ratio exhibited formation of discs with significantly larger diameters than wild type, accelerated retinal degeneration, and decreased physiological function. In contrast, models with a decreased ratio display properly oriented rod outer segments (ROSs) with decreased diameters but possess reduced sensitivity and faster flash-response kinetics before degenerating over a slow time course. Through the utilization of a mouse model that expresses approximately half the normal levels of RHO, we successfully alleviated the stress associated with abnormally high RHO: PRPH2 ratio, which emphasized the significance of maintaining this ratio for proper photoreceptor structure and function. It is imperative to acknowledge that any therapeutic interventions targeting PRPH2-affected photoreceptors must focus on maintaining the RHO: PRPH2 ratio as close as it is in wild type retinas. These findings also shed light on the delicate nature of rod photoreceptors, indicating that their heightened sensitivity to excessive opsin levels. Pathogenic variants in PRPH2 are known to decrease the levels of functional protein contributing to the formation of the elongated discs.

Given the absence of approved treatments, the presence of numerous low-prevalence pathogenic variants, and the involvement of intricate pathogenic pathways, it is imperative to identify a universally and effective therapeutic target for PRPH2 pathogenic variants.

This invention was made with government support under grant No. NIH EY10609 awarded by the National Institute of Health. The government has certain rights in the invention.

### SUMMARY

The present disclosure relates to methods of treating peripherin-2 (PRPH2) related retinal disease by administering a therapeutic treatment to downregulate the expression of rhodopsin (RHO).

Modifying the RHO/PRPH2 ratio in favor of PRPH2 positively impacts the disease phenotype in previously characterized knockin mouse models expressing the patient pathogenic variants p.Lys154del (c.458-460del) (Prph2^{K153Δ/+}) and p.Tyr141Cys (c.422A>G) (Prph2^{Y141C/+}) in PRPH2. Studies described below revealed that genetic ablation of one allele of Rho in the heterozygous models improves photoreceptor ultrastructure and physiological function. Furthermore, in order to demonstrate the translational applicability of this strategy, a previously characterized antisense oligonucleotide (ASO) mRho ASO1 shown to effectively reduce Rho transcript levels was employed following intravitreal administration. ASO-mediated reduction of RHO levels in mice heterozygous for the Prph2 Y141C knockin mutation resulted in improved retinal function, OS ultrastructure, and delayed photoreceptor degeneration. Thus, reducing RHO levels is an effective therapeutic strategy to ameliorate the disease phenotype in patients with PRPH2-associated inherited retinal disorders.

### BRIEF DESCRIPTION OF DRAWINGS

FIG. 1 shows representative (A) scotopic and (B) photopic ERG waveforms at P30 from all genotypes in Prph2^{Y141C/+} and Prph2^{K153A/+} mice following partial ablation of Rho and (C-F) mean maximum amplitudes of scotopic a-waves and b-waves, as well as photopic b-waves.

FIG. 2 shows (A) representative images of H&E stained retinal sections at P30 and P90 and (B-C) nuclei counts performed in 100 μm width windows in retinal sections taken from the indicated genotypes in Prph2^{K153N/+} or Prph2^{Y141C/+} following partial ablation of Rho.

FIG. 3 shows (A) representative TEM images at low and high-magnification of tannic acid/uranyl acetate-stained retinas from WT, Rho^{+/-}, Prph2^{Y141C/+}, Prph2^{Y141C/+}/Rho^{+/-}, Prph2^{K153Δ/+}, and Prph2^{K153N/+}/Rho^{+/-} at P16, (B) quantification of open discs at the base of the OS in the listed genotypes at P16, utilizing data from a previous publication for WT and Prph2^{Y141C/+} open discs, and (C) quantification of OS diameters measured in the listed genotypes at P16, with each data point representing a single outer segment.

FIG. 4 shows (A) representative immunodot blot images of retinal extracts taken at P30 from the listed genotypes in Prph2^{Y141C/+} and Prph2^{K153Δ/+} retinas, and signal intensity measurement of the dots in the immunoblots for the listed genotypes at P30 and probed for (B) RHO and (C) PRPH2 normalized to actin and plotted relative to WT.

FIG. 5 shows (A) representative immunoblots from P30 retinal extracts from the indicated genotypes and separated on SDS-PAGE, under non-reducing conditions, and (B-C) percent of total intensities of monomers, dimers and high order complexes for each genotype were plotted as mean±SD for PRPH2 and ROM1.

FIG. 6 shows (A) representative immunostainings of P30 retinal sections from the indicated genotypes stained for GFAP and DAPI, (B) representative immunodot blots probed for GFAP (left) and actin (right), and (C) fold changes in GFAP relative to WT quantified from the immunodot blots presented in B and plotted as mean±SD.

FIG. 7 shows (A) design of the titration studies for the early-stage preclinical mRho ASO1 dosage in Prph2^{Y141C/+} mice, (B-C) scotopic a-, scotopic b-, and photopic b-wave amplitudes plotted as mean±SD of the percent of the independent vehicle control for early intervention dosage titrations measured at P60, and (D-E) scotopic a-, scotopic b-, and photopic b-wave amplitudes plotted as mean±SD of the percent of the independent vehicle control eye for early intervention dosage titrations measured at P90.

FIG. 8 shows (A) design of preclinical late-stage dosage titration studies with mRho ASO1 in Prph2^{Y141C/+} mice, (B-C) scotopic a-, scotopic b-, and photopic b-wave amplitudes plotted as mean±SD of the percentage of the contralateral vehicle control for late intervention dosage titrations measured at P60, and (D-E) scotopic a-, scotopic b-, and photopic b-wave amplitudes plotted as mean±SD of the percentage of the contralateral vehicle control eye for late intervention dosage titrations measured at P90.

FIG. 9 shows (A) schematic representation of the design for injection of the control ASO and the functional assessments of ERG responses of injected eyes, (B) representative waveforms of scotopic and photopic responses recorded 15 days post-injection of the control ASO, and (C) mean±SD maximum amplitudes of scotopic a- and b-waves and photopic a- and b-waves of control ASO treated eyes relative to either untreated or vehicle treated eyes.

FIG. 10 shows (A) schematic representation of the design for injection of the control ASO and the histologic evaluations, (B) representative light images of retinal cross sections from eyes injected with control ASO, compared to either untreated or vehicle-injected eyes, 15 days post-injection, and (C) spidergram representing the count of photoreceptor nuclei in the outer nuclear layer of eyes injected with control ASO, compared to vehicle-injected and untreated eyes.

FIG. 11 shows (A) schematic representation of the design for injection of the control ASO and the immunodot analyses, (B) representative immunodot blots used to assess the levels of RHO and PRPH2 in retinal extracts from eyes injected with control ASO, in comparison to untreated or vehicle-injected eyes, 15 days post-injection, and (C) quantification of the levels of RHO (upper panel) or PRPH2 (lower panel) in retinal extracts from eyes injected with control ASO, in comparison to vehicle-injected or untreated eyes.

FIG. 12 shows (A) schematic representation of the design for early-stage preclinical mRho ASO1 intervention studies in Prph2^{Y141C/+} mice at PI-15, PI-45 and P75, (B) representative waveforms of scotopic and photopic responses recorded 15 days after injection at P30, and mean±SD maximum amplitudes of (C) scotopic a-waves and scotopic b-wave and (D) photopic a-wave and b-wave amplitudes of treated (3.125 μg mRho ASO1) and untreated contralateral control eyes recorded in preclinical early injection studies at ages P30, P60, and P90.

FIG. 13 shows (A) design of late-stage preclinical intervention studies with mRho ASO1 in Prph2^{Y141C/+} mice, (B) representative scotopic and photopic waveforms recorded at 15 and 45 days post injections at P45, and (C-D) mean maximum amplitudes of scotopic a-waves and b-waves, as well as photopic a-waves and b-waves were plotted as mean±SD for treated eyes (6.25 μg mRho ASO1) and vehicle control contralateral eyes in the late-stage intervention studies conducted at P60 and P90.

FIG. 14 shows quantification of (A) RHO and (B) PRPH2 in retinal extracts at P30 (PI-15), P60 (PI-45), and P90 (PI-75) after injections with mRho ASO1 at P15 (3.125 μg mRho ASO1), (C) graphs depicting the ratio of RHO to PRPH2 following P15 injection, determined by dividing the RHO signal intensity value by that of PRPH2, Quantification of (D) RHO and (E) PRPH2 in retinal extracts at P60 (PI-15) and P90 (PI-45) after injections with mRho ASO1 at P45 (6.25 μg mRho ASO1), (F) graphs illustrating the ratio of RHO to PRPH2 following P45 injections, transcript levels assessed by qRT-PCR at PI-15, with quantification of (G) Rho and (H) Prph2 mRNA transcript levels relative to Gapdh for P15 and P45 injected samples, and (I) quantification of Rho mRNA transcript levels relative to Prph2 for P15 and P45 injected samples.

FIG. 15 shows (A) representative images of retinal sections stained with H&E at P90 after early-stage treatment with mRho ASO1 in Prph2^{Y141C/+} mice, (B-D) nuclear counts from 100 μm-windows at every 500 μm distance from the optic nerve and across the superior-inferior plane of retinal sections.

FIG. 16 shows representative TEM images captured from retinas at P60 following mRho ASO1 intervention showing (A) untreated contralateral control and (B) 3.125 μg mRho ASO1 injected eyes at P15 and evaluated at P60 (PI-45).

FIG. 17 shows representative TEM images of retinas at P90 following mRho ASO1 intervention showing (A) untreated contralateral eyes and (B) 6.25 μg mRho ASO1 treated eyes at P45 and evaluated at P90 (PI-45).

FIG. 18 shows (A) representative low-magnification TEM images of tannic acid/uranyl acetate-stained retinas from mRho ASO1 treated eyes 45 days following treatment at P15 (3.125 μg mRho ASO1) and P45 (6.25 μg mRho ASO1) and untreated contralateral control eyes, and (B) a representative image of a whorl-like structure present in P60 uninjected contralateral Prph2^{Y141C/+} eye (left) and quantification of whorls presented as a percentage of the total number of counted OSs. N=144-307 OSs counted per retina (right).

FIG. 19 shows representative TEM images of tannic acid/uranyl acetate-stained retinas from Prph2^{Y141C/+} eyes with control injection of PBS at P45 and collected at P90 for (A) Mononucleated cell located in the subretinal space, (B) extended processes observed as a characteristic of these cells, and (C) cells found to possess a large amount of phagocytosed material.

FIG. 20 shows (A) representative high-magnification TEM images of tannic acid/uranyl acetate-stained retinas taken from P90 mRho ASO1 treated and contralateral control eyes that were injected at P15 (3.125 μg mRho ASO1) and P45 (6.25 μg mRho ASO1), and quantification of (B) open discs at the base of the OS and (C) OS diameters in mRho ASO1 injected and contralateral control eyes 45 days after treatment.

### DETAILED DESCRIPTION OF PREFERRED EMBODIMENTS

The present disclosure relates to a novel therapeutic strategy targeting the ratio between rhodopsin (RHO) and peripherin-2 (PRPH2) to address pathological structural phenotype.

Photoreceptors exhibit high metabolic demands and are prone to the accumulation of toxic photo-oxidative products associated with the visual cycle. To ensure their proper function and overall health, photoreceptors undergo a daily physiological renewal process that is crucial. This process involves diurnal shedding of the distal portion of the outer segment, where discs are phagocytosed by the RPE, followed by the formation of new disc at the proximal end to replace them. The presence of PRPH2 is crucial for proper disc morphogenesis, as its absence leads to failure in outer segment formation and the release of membrane evaginations from the connecting cilium in the form of ectosomes. PRPH2 plays a dual role, not only inhibiting ectosome release but also participating in the proper enclosure of mature photoreceptor discs. Defects in discs enclosure lead to misaligned overgrown discs and aberrations in the form of whorls, which are characteristics features observed in photoreceptor outer segments of animal models expressing Prph2 pathogenic variants. These structural abnormalities in the outer segments significantly impact photoreceptor function, leading to progressive loss of visual function and a reduction in the number of viable photoreceptor cells. Despite the identification of numerous pathogenic variants in the PRPH2 gene in patients, finding an effective therapeutic approach remains challenging.

As discussed below, partial genetic ablation of Rho was used to modulate protein levels in two well-established mouse models of patient pathogenic variants (Prph2^{Y141C/+} and Prph2^{K153N/+}). This reduction in RHO levels resulted in improved maximum physiological responses driven by rods in both models due to enhanced rod outer segment structure. Interestingly, despite being a rod-specific protein, the decrease in RHO reduction also improved cone-driven responses in both models. This finding is in line with the well-documented symbiotic relationship between rods and cones, where cone degeneration is often secondary to rod degeneration in retinitis pigmentosa. The proper alignment of rod outer segment may contribute to the upright positioning of cone outer segments, resulting in enhanced responses to light stimulation. Previous studies have demonstrated that rods produce factors supportive of cones. It is conceivable that mRho ASO treatment improved rod outer segment structures, thereby reducing cellular stress and promoting the production of these supportive factors, consequently leading to improved cone responses.

Transient improvements in physiological function were observed for Prph2^{K153N/+}, supporting RHO reduction as a therapeutic strategy for this mutation. Previous efforts to supplement the Prph2 gene in Prph2^{K153N+} mice by crossing them with a PRPH2-overexpresser line demonstrated limited efficacy, resulting in minimal improvements in scotopic a-wave ERGs at P30. Therefore, the transient rescue of scotopic a-, scotopic b-, and photopic b-wave maximum amplitudes associated with RHO reduction is significant.

To demonstrate the clinical relevance of this strategy and achieve controlled reduction of RHO levels, a previously characterized ASO was used, mRho ASO1. This ASO has been employed as a control and has demonstrated specificity for wild type RHO. ASOs are single-stranded nucleic acids with chemically modified backbones designed to bind to their target mRNA and regulate protein expression. Traditionally, this heteroduplex formation aims to decrease aberrant protein expression through various mechanisms, including RNase H mediated cleavage of the heteroduplex, splicing modulation, and steric hindrance of ribosomal binding. ASOs have shown a good safety profile in numerous clinical trials, resulting in 10 FDA approved therapeutics. The specific oligonucleotide, mRho ASO1, has the added benefit of previously demonstrating in vivo efficacy in reducing Rho mRNA in a dose-dependent manner following intravitreal injection. Intravitreal administrations have become a standard procedure for the treatment of retinal diseases since the first FDA approved intravitreal injection therapeutic in 1998. With millions of intravitreal treatments performed annually, significant advancements have been made in injection-related procedures and tools to minimize patient discomfort and injection complications, making intravitreal injection the current preferred method of delivery for retinal disease therapy.

Two intervention time points were evaluated, considering the wide variability in age of onset and severity for PRPH2 associated phenotypes. For these experiments, Prph2^{Y141C/+} mice, known for their slower rate of degeneration, were selected, allowing for a longer window of intervention and assessment. Early intervention was initiated shortly after mice open their eyes at P15, while late intervention took place at P45 after full development of the retina and the initiation of functional decline in rods and cones. Both early and late therapeutic interventions resulted in improved scotopic and photopic maximum a-wave responses. While it was expected that earlier intervention would yield superior functional rescue, the maximum a-wave values were similar between early and late intervention when evaluated at P60 and P90. No significant changes in maximum b-wave amplitudes were observed following mRho ASO1 treatment, despite observing improvements in b-wave responses following partial genetic ablation of RHO in Prph2^{Y141C/+} mice.

In addition to the observed improvements in ERG responses, early mRho ASO1 intervention led to significant increases in the number of photoreceptor nuclei. By administering the mRho ASO1 to modulate protein levels, RHO reduction was optimized to achieve the proper RHO: PRPH2 ratios, resulting in maximum physiological response improvements. This dosage preserved the photoreceptors and slowed down the rate of degeneration. An inadequate reduction of RHO would lead to insignificant changes and minimal preservation, while excessive reduction of RHO would worsen photoreceptor death and degeneration. Late stage mRho ASO1 intervention failed to delay photoreceptor cell death. It is possible that, despite optimization for maximum functional improvement, P45 might be beyond the threshold of disease progression for mRho ASO1 to effectively improve photoreceptor survival. These findings further underscore the importance of maintaining proper RHO: PRPH2 ratios and choosing the appropriate time point for intervention to effectively prevent photoreceptor death using the reduction of RHO as a strategy.

In line with observations in the Prph2 double mutants, ultrastructural analyses revealed that mRho ASO1 therapy improved rod outer segment ultrastructure throughout the retina, as evidenced by a reduction in large membranous whorls and sagittally oriented discs. Additionally, reduction of RHO following mRho ASO1 treatment reduced the structural abnormalities of increased open nascent discs and outer segment diameters associated with Prph2 pathogenic variants. Furthermore, a decrease in microglia infiltration following late stage mRho ASO1 intervention at P90 was observed. As microglia infiltration is often observed in photoreceptor degenerative conditions, this decrease further illustrates the overall benefit of mRho ASO1 on overall photoreceptor health and structure.

Taken together, these results establish the therapeutic efficacy of reducing levels of RHO as a strategy in impeding retinal degeneration caused by PRPH2 pathogenic variants. The Prph2^{Y141C/+} knockin model has been shown to recapitulate many known patient phenotypes, including photoreceptor structural defects, decreased scotopic and photopic ERG values, and fundus flecking. The strategy of reducing RHO levels can serve as a preventative therapeutic approach for individuals with PRPH2 mutations.

Methods for downregulation of rhodopsin as a therapeutic strategy for PRPH2-associated disease are disclosed herein. Preferred embodiments for the downregulation of rhodopsin utilize antisense oligonucleotides (ASO) that lead to producing less rhodopsin such as by degradation of rhodopsin mRNA.

In additional preferred embodiments a 20-mer antisense oligonucleotide named mRho ASO1 is used. It is a chimeric 20-mer single stranded DNA molecule with a sequence of 5′-AGCTACTATGTGTTCCATTC-3′ (SEQ ID NO:1). It contains a phosphorothioate backbone with wings containing 2′-O-methoxyethyl modifications at positions 1-5 and 15-20. It is complimentary to a target sequence in exon 5 of rhodopsin mRNA leading to its degradation. This mRNA degradation results in less rhodopsin protein produced. Downregulating rhodopsin using—for example—mRho ASO1 is efficacious at delaying disease progression in peripherin-2 associated disease.

The current invention also pertains to methods of prevention or therapy for PRPH2 related diseases, including the step of administering a composition that inhibits the production of rhodopsin in accordance with preferred embodiments disclosed herein. In preferred embodiments, the methods of prevention or therapy for diseases relating to PRPH2 include the step of administering a composition comprising of an antisense oligonucleotide (ASO) that inhibits the expression of the full complement of rhodopsin in a subject in need of such therapy.

In another aspect of the present invention there is provided a therapeutic composition including a therapeutically effective amount of a composition that inhibits the production of rhodopsin as defined above and a pharmaceutically acceptable excipient, adjuvant, carrier, buffer or stabilizer. A “therapeutically effective amount” is to be understood as an amount of an exemplary composition that is sufficient to show inhibitory effects on the production of rhodopsin. The actual amount, rate and time-course of administration will depend on the nature and severity of the disease being treated. Prescription of treatment is within the responsibility of general practitioners and other medical doctors. The pharmaceutically acceptable excipient, adjuvant, carrier, buffer or stabiliser should be non-toxic and should not interfere with the efficacy of the active ingredient. The precise nature of the carrier or other material will depend on the route of administration, which may by injection or extraocular.

In preferred embodiments, the composition is administered by intravitreal injection. Additional preferred routes of administration include extraocular delivery such as by the use of eye drops.

In another aspect, there is provided the use in the manufacture of a medicament of a therapeutically effective amount of composition as defined above for reducing the production of rhodopsin for administration to a subject.

The term “pharmacologically acceptable salt” used throughout the specification is to be taken as meaning any acid or base derived salt formed from hydrochloric, sulfuric, phosphoric, acetic, citric, oxalic, malonic, salicylic, malic, fumaric, succinic, ascorbic, maleic, methanesulfonic, isoethonic acids and the like, and potassium carbonate, sodium or potassium hydroxide, ammonia, triethylamine, triethanolamine and the like.

The term “prodrug” means a pharmacological substance that is administered in an inactive, or significantly less active, form. Once administered, the prodrug is metabolised in vivo into an active metabolite.

The term “therapeutically effective amount” means a nontoxic but sufficient amount of the drug to provide the desired therapeutic effect. The amount that is “effective” will vary from subject to subject, depending on the age and general condition of the individual, the particular concentration and composition being administered, and the like. Thus, it is not always possible to specify an exact effective amount. However, an appropriate effective amount in any individual case may be determined by one of ordinary skill in the art using routine experimentation. Furthermore, the effective amount is the concentration that is within a range sufficient to permit ready application of the formulation so as to deliver an amount of the drug that is within a therapeutically effective range.

Further aspects of the present invention will become apparent from the following description given by way of example only.

### EXAMPLES

### Materials and Methods

### Study Approvals and Generation/Acquisition of Mutant Lines

The knockin mouse models, Prph2^{K153Δ/K1534} and Prph2^{Y141C/Y141C}, were generated and characterized previously. Additionally, an in-house WT mouse strain was generated as described previously. The homozygous Rho knockout mouse line (Rho-) was originally obtained as a generous gift and has been previously described. Knockin heterozygotes (Prph2^{K153A/+} and Prph2^{Y141C/+}) mice were generated by crossing the WT mice with either Prph2^{K153Δ/K1534} or Prph2^{Y141C/Y141C} mice, respectively. Double heterozygotes (Prph2^{K153Δ/+}/Rho^{+/-} and Prph2^{Y141C/+}/Rho^{+/-}) were produced through crossbreeding of Prph2^{K153Δ/K1534} or Prph2^{Y141C/Y141C} mice with Rho^{-/-} mice. All mice were raised under cyclic light conditions, with a 12 h-light/12-dark cycle (12L/12D), and an illuminance of 30 lux. Mice were euthanized by carbon dioxide asphyxiation followed by cervical dislocation. Both mouse sexes were used in this study and no differences were observed between them.

### Electroretinography

Full field scotopic and photopic electroretinograms (ERGs) were recorded as described before, with minor modifications using a UTAS system (LKC; Gaithersburg, MD). Eyes of overnight dark-adapted mice were dilated with 1% cyclopentolate solution (Bausch+Lomb; Bridgewater, NJ) prior to inducing anesthesia via intramuscular injection of 85 mg/kg ketamine (Covetrus; Portland, ME) and 14 mg/kg xylazine (Akorn, Inc.; Lake Forest, IL). The reference needle electrode was placed subdermally between the ears instead of the cheek as previously described. Values obtained for ERG experiments using the double heterozygote animals were averaged and plotted. For injected animals, following the recording of the initial values, the probes were replaced, and a second measurement was taken to ensure observed trends were accurate. Only the initially recorded values were used for analysis.

### Histology and Morphometry

Histology and morphometric analyses were performed as previously described. Briefly, eyes were enucleated from euthanized mice and fixed in modified Davidson's fixative (12% formaldehyde, 15% ethanol, and 5% glacial acetic acid) overnight at 4° C. Fixed eyes were then dehydrated and embedded in paraffin before being sectioned at 10 μm thickness. Sections containing the optic nerve head were stained with hematoxylin and eosin (H&E) (Sigma Aldrich, Burlington, MA, MHS16 and HT110116) and mounted with Permount (Fisher Scientific, Waltham, MA, P15-100) mounting medium. For morphometric analysis, 100 μm width window images were captured every 500 μm starting at the optic nerve head using a Zeiss Axioskop and a 20× objective lens. Image J was used for morphometric analysis by manually counting the number of nuclei in the 100 μm window images captured at the indicated distances. Representative images were taken using a 40× objective.

### Protein Extraction and Quantification

Retinas from the indicated genotypes and treatment groups were extracted from euthanized mice and immediately flash frozen using liquid nitrogen before being stored at −80° C. until processing. Retinas were homogenized and then sonicated in 50 μL 1X RIPA Buffer (ABCAM, Boston, MA, ab156034) per retina. Samples were then incubated in the same extraction buffer over night at 4° C. Following extraction, samples were centrifuged at 18,200× g for 15 minutes at 4° C. to remove insoluble debris. Extracted protein concentrations were measured using the colorimetric Bradford Assay (Bio-Rad, Hercules, CA, #5000006).

Immunodot blots were used to quantify protein levels. Extract titrations were performed to determine the optimal range of total protein level needed from each genotype prior to quantification experiments. A MINIFOLD I microsample filtration manifold (Whatman® Schleicher & Schuell®, Keene, NH #27510) was used to load the protein extracts onto a nitrocellulose membrane (Bio-Rad, Hercules, CA #1620112). The membrane was allowed to dry before blocking with 5% non-fat milk in 1X TBST (0.1% Tween®). Following blocking, membranes were incubated with either unconjugated primary antibody (anti-RHO, -PRPH2, or GFAP) or anti-actin primary antibody conjugated with Horseradish Peroxidase (HRP) at room temperature. Membranes were washed prior to incubation with the appropriate HRP-conjugated secondary antibodies at room temperature. Following a final wash, the membrane was incubated with ECL reagent (SuperSignal™ West PICO PLUC Chemiluminescent Substrate #34577) for 1 minute, chemiluminescence imaging was performed using ChemiDoc™ MP imaging system (Bio-Rad, Hercules, CA), and quantification was performed using Image Lab software v6.0.1 (Bio-Rad).

For protein quantification using immunodot blot, three to five replicates were used for each genotype and for each treatment. Each sample was independently measured in triplicate on each blot, averaged to be considered one value and presented as a mean±SD. For all immunoblotting quantification involving double heterozygote mice, chemiluminescent signal intensity values obtained for PRPH2, RHO, and GFAP were normalized by β-actin signal intensity before further normalization to the internal WT control. Quantification for the ASO treatment experiments involved normalization of measured RHO and PRPH2 chemiluminescent signal intensity values by β-actin signal intensity to obtain RHO/β-actin and PRPH2/β-actin values. Additionally, RHO was normalized by PRPH2 in order to obtain RHO: PRPH2 ratios.

Protein oligomerization was analyzed by performing non-reducing sodium dodecyl sulfate-polyacrylamide gel electrophoresis (SDS-PAGE) followed by immunoblotting using previously described methods. Chemiluminescent signal intensity values obtained for each band were divided by the total lane signal intensity to obtain the percent distribution.

### Transmission Electron Microscopy (TEM)

Fixation and processing of mouse eyes for TEM was performed as described previously. In short, anesthetized mice were transcardially perfused with 2% paraformaldehyde, 2% glutaraldehyde and 0.05% calcium chloride in 50 mM MOPS (pH 7.4). Eyes were post-fixed for an additional 2 hour. Eyecups were embedded in 2.5% low-melt agarose (Precisionary Instruments, Greenville, NC) and sectioned on a Vibratome (VT1200S; Leica, Buffalo Grove, IL). 200 μm agarose sections were stained with 1% tannic acid (Electron Microscopy Sciences, Hatfield, PA) and 1% uranyl acetate (Electron Microscopy Sciences). Stained sections were gradually dehydrated with ethanol and infiltrated and embedded in Spurr's resin (Electron Microscopy Sciences). 70 nm plastic sections were cut, placed on copper grids and stained with 2% uranyl acetate and 3.5% lead citrate (19314; Ted Pella, Redding, CA). Samples were imaged on a JEM-1400 electron microscope (JEOL, Peabody, MA) at 60 kV with a digital camera (BioSprint; AMT, Woburn, MA). Image analysis and processing was performed with ImageJ.

mRho ASO1 Synthesis

A previously characterized Rho-specific ASO mRho ASO1 targeting exon 5 (5′-AGCTACTATGTGTTCCATTC-3′) (SEQ ID NO: 1) containing a phosphorothioate backbone and 2′-O-methoxyethyl modifications at nucleotides 1-6 and 15-20 was used. Control (5′-CCTATAGGACTATCCAGGAA-3′) (SEQ ID NO: 2) and mRho ASO1 antisense oligonucleotides were synthesized by Integrated DNA Technologies, Inc. (Coralville, Iowa). Synthesis was performed according to their standard operating procedures using proprietary processes.

### Intravitreal Injections

Intravitreal injections were performed as previously described. Briefly, Prph2^{Y141C/+} mice were injected intravitreally at P30 for control ASO, P15 for early-stage intervention and P45 for late-stage intervention. Eyes were dilated with 1% cyclopentolate solution (Bausch+Lomb; Bridgewater, NJ) for 5 minutes prior to intramuscular injection of 85 mg/kg ketamine (Covetrus; Portland, ME) and 14 mg/kg xylazine (Akorn, Inc.; Lake Forest, IL). Adequate anesthesia was confirmed prior to injection by pedal withdrawal reflex assessment. A 30 gauge sterile needle was used to puncture the cornea and provide a guide hole. A Hamilton syringe was inserted through the guide hole to manually dispense material [mRho ASO1 or Endotoxin-Free Dulbecco's PBS (1X) (EMD Millipore; Billerica, MA)] into the vitreal chamber. Volumes injected for all studies were 0.5 μL in P15 animals and 1.0 μL in P45 animals. Dosages used for P15 titration studies included 1.5625 μg, 3.125 μg, 6.25 μg, 12.5 μg, and 25 μg, while dosages used for P45 titrations included 3.125 μg, 6.25 μg, 12.5 μg, 25 μg, and 50 μg. Following dosage titration studies, a concentration of 6.25 μg/μL was used for all injections so that P15 mice received 3.125 μg mRho ASO per injection while P45 mice received 6.25 μg mRho ASO per injection. Following injection, Triple antibiotic ointment (Taro pharmaceuticals Inc., Hawthorne, NY, USA) was applied to each eye. All animals were closely monitored during recovery from anesthesia until fully ambulatory and provided adequate access to food and water. Mice experiencing injection related complications, such as significant accumulation of blood in the vitreous chamber, retinal detachment, damage to the iris or lens, intraocular infection, or cataract development were excluded from the study. All injections were performed in a sterile surgical suite. In the titration studies, both eyes were injected for all mice with either mRho ASO1 or 1X PBS vehicle control. Due to procedure related difficulties experienced during the titration studies when attempting to inject both eyes at P15, all characterization studies were performed injecting one eye with 3.125 μg ASO and the other serving as an uninjected contralateral control. All P45 characterization studies were performed using the same procedure as titration studies with 6.25 μg ASO.

### Immunofluorescence

Immunofluorescence was performed as previously described. Briefly, mice were euthanized by carbon dioxide asphyxiation followed by cervical dislocation, eyes were enucleated, and then fixed in modified Davidson's fixative overnight at 4° C. Fixed eyes were then dehydrated and embedded in paraffin before being sectioned at 10 μm thickness. Before use, samples were incubated in xylene to remove paraffin and tissue was rehydrated in sequential ethanol dilutions. Following rehydration, antigen retrieval was performed by incubating samples in sodium citrate at 100° C. for 30 minutes before being placed in blocking buffer (5% bovine serum albumin, 1% donkey serum, and 0.5% Triton X-100 in PBS) for 1 hour. Following blocking, antigen labeling was performed by incubating sections with primary antibodies overnight, washing with 1×PBS, incubating with secondary antibodies for 2 hours, and then washing with 1×PBS. DAPI staining was then performed for 15 minutes at a concentration of 0.1 μg/mL (Thermo Fisher Scientific, Waltham, MA, USA, 62248). Slides were mounted using anti-fade mounting media and sealed with clear nail polish. Imaging was performed using a 20× objective on a Zeiss LSM800 confocal microscope (Zeiss, White Plains, NY, USA). Image processing was performed in Zen 3 lite software.

### Antibodies

All antibodies used for immunoblotting and immunofluorescence are listed below in Table 1.

TABLE 1

| | | | Application/ | |
|---|---|---|---|---|
| Antigen | Species | Clone | Concentration | Source |
| PRPH2 | Mouse | 2B7 | 1:1,000 (IB) | Millipore |
| ROM1 | Mouse | 2H5 | 1:1,000 (IB) | Millipore |
| RHO | Mouse | 1D4 | 1:2,000 (IB) | Santa Cruz |
| | | | | Biotechnology |
| GFAP | Mouse | GA5 | 1:1,000 (IB) 1:500 (IF) | Sigma-Aldrich |
| Actin-HRP | Mouse | AC-15 | 1:15,000 (IB) | Sigma-Aldrich |
| Mouse | Goat | | 1:15,000 (IB) | Millipore |
| IgG HRP | | | | |

### Statistical Analysis

Statistical Analysis was performed using GraphPad Prism version 8 (GraphPad Software, La Jolla, CA). One-way ANOVA with Tukey's post-hoc comparisons was employed for quantitative analysis between groups for all double mutant ERG data and comparison of protein levels in double mutant figures. Two-way ANOVA with Tukey's post-hoc comparisons was employed for quantitative analysis between groups for all morphometry analysis. ASO treated ERGs were compared using two-tailed Mann-Whitney U test. Student's two-tailed t-test was used for statistical analysis of protein quantification studies. Significance was set at P<0.05. Pairwise comparison significances are indicated as *P<0.05, ** P<0.01, *** P<0.001, **** P<0.0001 throughout the text.

### Results

### Reducing RHO Levels in Prph2 Knockin Mouse Models of Human PRPH2 Pathogenic Variant Leads to Functional Improvements

In Rho^{+/-} mice, retinal development proceeds normally with proper lamination but gradually undergoes degeneration over time. The reduction of RHO manifests specific effects on the ultrastructure of ROS, including an elliptical disk shape and decreased surface area. To determine the impact of reducing RHO levels in ROS discs on functionality of the retinas from mice expressing mutant forms of PRPH2, comprehensive electroretinography measurements (ERG) were conducted on Prph2^{K153Δ/+} and Prph2^{Y141C/+} mice that were also hemizygous for Rho (Rho^{+/-}). These Prph2 heterozygous mice were chosen for their clinical relevance, as PRPH2-associated retinitis pigmentosa typically exhibits autosomal dominant inheritance. ERGs were performed at various postnatal (P) stages (P17, P30, and P90) to evaluate differences in scotopic and photopic maximum amplitudes during photoreceptor maturation and disease progression. Results showed improved rod and cone functions in Prph2^{Y141C/+} and Prph2^{K153Δ/+} mice following partial ablation of Rho.

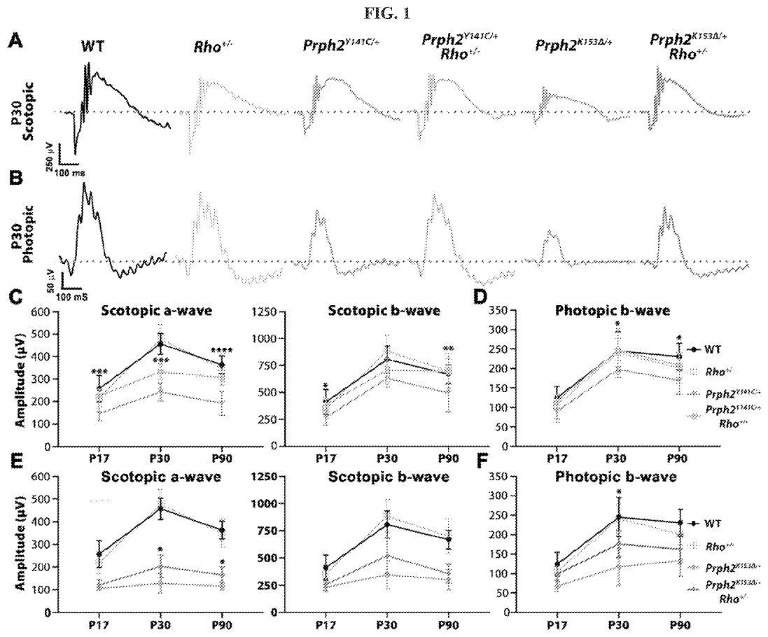

FIG. 1 shows representative (A) scotopic and (B) photopic ERG waveforms at P30 from all genotypes and (C-F) mean maximum amplitudes of scotopic a-waves and b-waves, as well as photopic b-waves, plotted as mean±SD at P17, P30, and P90. * P<0.05, ** P<0.01, *** P<0.001, **** P<0.0001 by one-way ANOVA with Tukey's post-hoc comparison. N=9-15 animals/genotype/age. In FIG. 1, * denotes comparisons between (C-D) Prph2^{Y141C/+} and Prph2^{Y141C/+}/Rho^{+/-} or (E-F) Prph2^{K153Δ/+} and Prph2^{K153Δ/+}/Rho^{+/-}.

Eliminating one allele of Rho in Prph2^{Y141C/+} mice led to a significant improvement in mean maximum amplitudes of scotopic a- and b-waves as early as P17 compared to Prph2^{Y141C/+} controls (~53% and ~43% increases respectively, FIG. 1C). Additionally, there was an increase in the photopic (~29%) b-wave mean maximum amplitude, although this increase did not reach statistical significance (FIG. 1D). These improvements persisted throughout the study, as Prph2^{Y141C/+}/Rho^{+/-} retinas exhibited significantly better mean maximum amplitudes of scotopic a-waves (~37%) and photopic b-waves (~25%) at P30, as well as scotopic a-waves (~60%) and b-waves (~40%) at P90 (FIG. 1, C and D).

For P17 Prph2^{K153Δ/+}/Rho^{+/-} mice, the assessment revealed modest non-significant functional improvements in the scotopic a- and b as well as photopic b-wave mean maximum amplitudes compared to Prph2^{K153N+} (~14%, ~10%, and ~45%, respectively) (FIG. 1, E and F). As the mice matured and their retinas fully developed, the partial ablation of Rho significantly enhanced scotopic a- and photopic b-wave amplitudes at P30 (~58% and ~51%, respectively). However, this statistically significant restorative effect was only apparent in the scotopic a-wave mean maximum amplitude (~42%) at P90 (FIG. 1, E and F).

### Reduced RHO Levels Result in Histologic Improvements

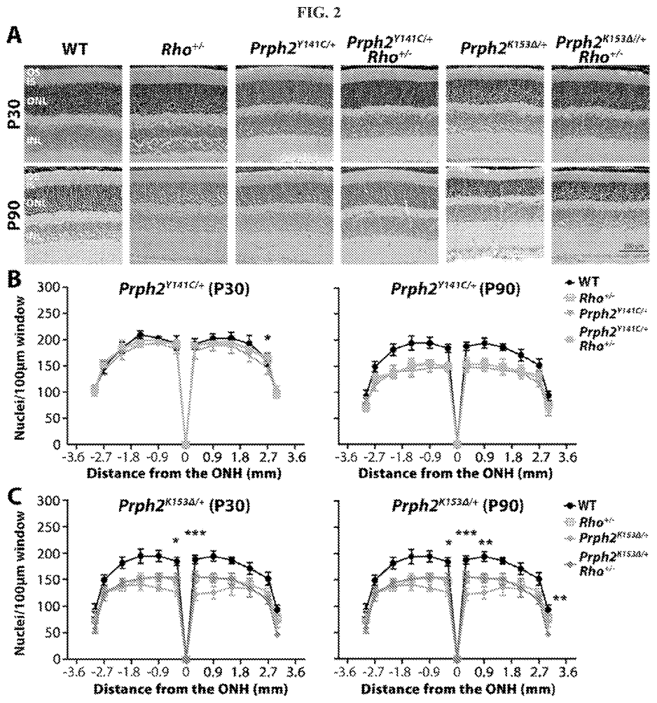

To determine if the observed functional improvements were attributed to increased photoreceptor survival, outer nuclear layer (ONL) counts were conducted for the different genotypes at P30 and P90. Despite the functional improvements, histologic and morphometric analyses at the light level revealed only minor changes between the Prph2 mutant models and their compound heterozygote/hemizygote counterparts. FIG. 2 shows that partial ablation of Rho does not affect ONL thickness in Prph2^{K153A/+} or Prph2^{Y141C/+}. FIG. 2 shows (A) representative images of H&E stained retinal sections at P30 and P90 and (B-C) Nuclei counts performed in 100 μm width windows in retinal sections taken from the indicated genotypes. Images were captured every 500 μm starting at the optic nerve head sections, and nuclei counts from multiple regions were plotted as mean±SD for the listed genotypes (n=3 for each genotype). * P<0.05, ** P<0.01, *** P<0.001 by two-way ANOVA with Tukey's post-hoc comparison. In FIG. 2, * denotes comparisons between (B) Prph2^{Y141C/+} or (C) Prph2^{K153Δ/+} and (B) Prph2^{Y141C/+}/Rho^{+/-} or (C) Prph2^{K153Δ/+}/Rho^{+/-}. Scale bar corresponds to 100 μm.

At P30, ONL counts were similar among WT, Rho^{+/-}, Prph2^{Y141C/+}, and Prph2^{Y141C/+}/Rho^{+/-} (FIG. 2(B), left). As the mice aged, significant loss of ONL nuclei was observed in Rho^{+/-}, Prph2^{Y141C/+}, and Prph2^{Y141C/+}/Rho^{+/-} compared to WT at P90 (FIG. 2(B), right). Modest ONL loss was noted in Prph2^{K153Δ/+} and Prph2^{K153Δ/+}/Rho^{+/-} at P30, progressing to significant photoreceptor loss in both models by P90 (FIG. 2(C)). However, a reduction in RHO levels was observed to improve average nuclear counts in the central regions of the Prph2^{K153Δ/+}/Rho^{+/-} retina at both time points (FIG. 2(C)).

### Reducing RHO Levels Leads to Enhanced Structural Integrity of the Outer Segments

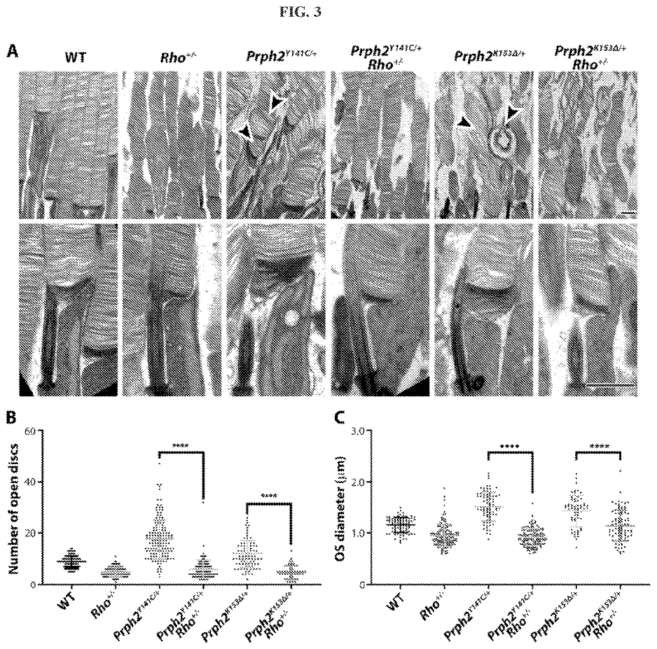

To explore additional factors contributing to the observed functional improvements, retinas underwent ultrastructural analyses. Prph2^{Y141C/+}/Rho^{+/-} and Prph2^{K153Δ/+}/Rho^{+/-} exhibit improved OS morphogenesis and ultrastructure compared to their respective controls. FIG. 3 shows (A) Representative TEM images at low and high-magnification of tannic acid/uranyl acetate-stained retinas from WT, Rho^{+/-}, Prph2^{Y141C/+}, Prph2^{Y141C/+}/Rho^{+/-}, Prph2^{K153Δ/+}, and Prph2^{K153Δ/+}/Rho^{+/-} at P16, (B) Quantification of open discs at the base of the OS in the listed genotypes at P16, utilizing data from a previous publication for WT and Prph2^{Y141C/+} open discs, and (C) Quantification of OS diameters measured in the listed genotypes at P16, with each data point representing a single outer segment. * P<0.05, ** P<0.01, *** P<0.001, **** P<0.0001 by one-way ANOVA (P<0.0001 for both open disc and OS diameter) with Tukey's post-hoc comparison. Arrows highlight misaligned, overgrown, and sagittally aligned discs, as well as whorl-like structures commonly observed in Prph2^{Y141C/+} and Prph2^{K153Δ/+} ROSs. N values for B: WT: 111, Rho^{+/-}: 112; Prph2^{Y141C/+}: 152; Prph2^{Y141C/+}/Rho^{+/-}: 105; Prph2^{K153Δ/+}: 98, Prph2^{K153Δ/+}/Rho^{+/-}: 43 and for C: WT: 86, Rho^{+/-}: 138, Prph2^{Y141C/+}: 80, Prph2^{Y141Δ/+}/Rho^{+/-}: 139, Prph2^{K153Δ/+}: 63, Prph2^{K153N/+}/Rho^{+/-}: 90. Scale bar, 1 μm. Error bars represent mean±SD.

Low magnification ultrastructure images revealed that the elimination of one allele of Rho resulted in an overall enhancement of OS structure in both Prph2 models (FIG. 3A, top panels). OSs display improved disc stacking and a reduction in the number of membranous whorls, particularly in Prph2^{Y141C/+}/Rho^{+/—} (arrowheads in FIG. 3A top panel).

Mutant PRPH2-associated defects in protein oligomerization have been linked to impaired disc closure, resulting in enlarged OS diameters and increased numbers of open, nascent discs. Tannic acid staining allows differentiation between nascent and mature discs, as the exposed nascent discs exhibit a darker staining pattern due to their exposure to the extracellular space. Higher magnification images were utilized to measure OS diameters and count open discs, providing a quantitative assessment of the impact of RHO reduction on these morphological abnormalities (FIG. 3A, lower panels). In the presence of WT PRPH2 levels, removing one allele of Rho decreased the average number of open nascent discs from ~9 in WT to ~5 in Rho^{+/-} (FIG. 3B). Similarly, the reduction in RHO levels resulted in decreased numbers of open discs, with averages of ~18 for Prph2^{Y141C/+} and ~12 for Prph2^{K153N/+} reduced to ~6 and ~5 in Prph2^{Y141C/+}/Rho^{+/-} and Prph2^{K153N/+}/Rho^{+/-}, respectively (FIG. 3B). The rescue effect was also evident in OS diameter measurements (μm), with mean values of 1.2, 1.5, and 1.4 in WT, Prph2^{Y141C/+}, and Prph2^{K153Δ/+}, respectively, reduced to 1.0, 0.9, and 1.1 in Rho^{+/-}, Prph2^{Y141C/+}/Rho^{+/-}, and Prph2^{K153Δ/+}/Rho^{+/-} (FIG. 3C). These findings demonstrate that improved ultrastructure of OSs underlies the observed functional improvements in Prph2^{Y141C/+}/Rho^{+/-} and Prph2^{K153Δ/+}/Rho^{+/-} mice.

### Partial Ablation of One Allele of Rho Leads to Reduced Levels of RHO Relative to PRPH2

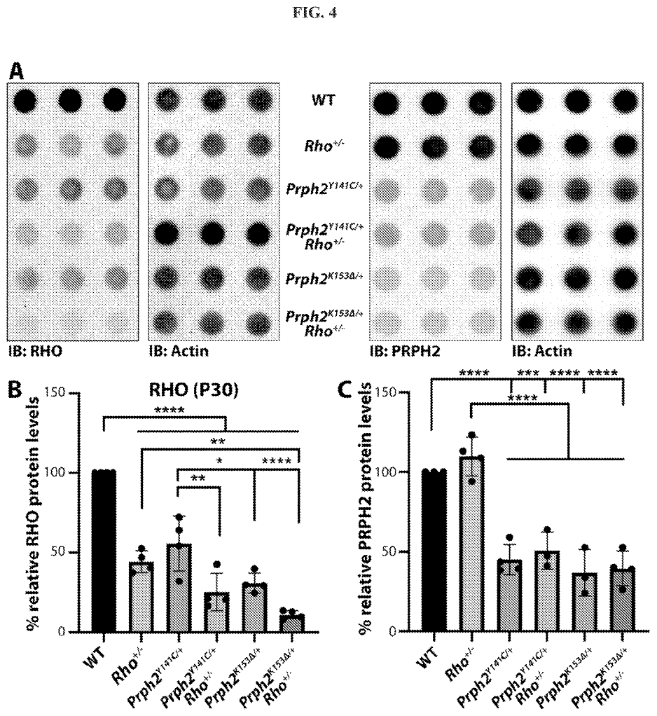

To establish the correlation between ablation of one Rho allele and the observed functional and structural improvements, immunodot blots were performed on retinal extracts from P30 mice. This aimed to determine the degree of decrease in RHO levels and the resulting RHO/PRPH2 ratio across all models. Results showed that partial ablation of Rho reduces the ratio of RHO to PRPH2 in Prph2^{Y141C/+} and Prph2^{K153Δ/+} retinas. FIG. 4 shows (A) Representative immune-dot blot images of retinal extracts taken at P30 from the listed genotypes, and (B-C) Signal intensity measurement of the dots in the immunoblots for the listed genotypes at P30 and probed for RHO (B) and PRPH2 (C) normalized to actin and plotted relative to WT. Data are presented as mean±SD. N=3-4 retinas/genotype. * P<0.05, ** P<0.01, *** P<0.001, **** P<0.001 by one-way ANOVA (P<0.0001 for both RHO and PRPH2) with Tukey's post-hoc comparison.

The partial genetic ablation of Rho resulted in reduction in protein levels in Rho^{+/-} (~56%), Prph2^{Y141C/+}/Rho^{+/-} (~55%), and Prph2K1534/+/Rho^{+/-} (~65%) compared to WT, Prph2^{Y141C/+}, and Prph2^{K1534/+}, respectively (FIG. 4(A)). Notably, this reduction did not impact the levels of PRPH2 (FIG. 4(C)). These findings highlight that the partial genetic ablation of Rho effectively decreases the ratio of RHO to PRPH2 in Rho^{+/-}, Prph2^{Y141C/+}/Rho^{+/-}, and Prph2^{K153Δ/+}/Rho^{+/-} retinas.

Partial Ablation of RHO does not Impact the Formation of Higher-Order Complexes of PRPH2

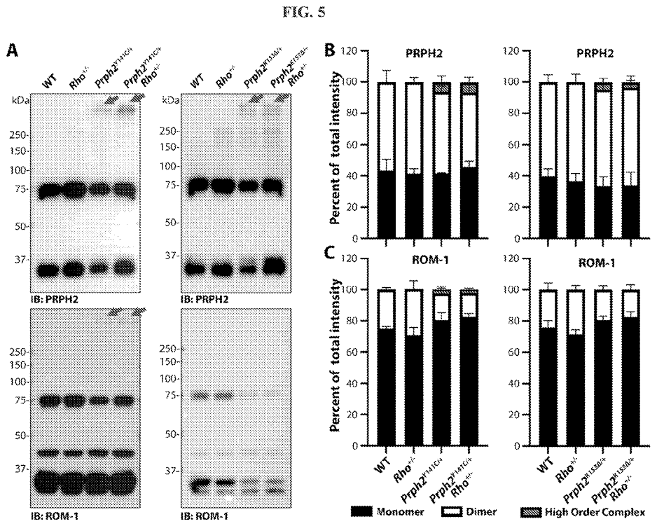

Since proper PRPH2 oligomerization is essential for disc formation and OS structure, the oligomerization status of PRPH2 in these mice following partial ablation of RHO was examined. Retinal extracts were subjected to immunoblotting after separation by sodium dodecyl sulfate-polyacrylamide gel electrophoresis (SDS-PAGE) under non-reducing conditions. Under these conditions, WT PRPH2 typically separates into monomers (~37 kDa) and disulfide-linked dimers (~75 kDa), while Prph2^{Y141C/+} and Prph2^{K153Δ/+} mutants have been shown to exhibit additional abnormal high-molecular weight protein complexes. Results show that partial ablation of Rho does not affect PRPH2/ROM1 large complex formation. FIG. 5 shows (A) Representative immunoblots from P30 retinal extracts from the indicated genotypes and separated on SDS-PAGE, under non-reducing conditions, and (B-C) Percent of total intensities of monomers, dimers and high order complexes for each genotype were plotted as mean±SD for PRPH2 and ROM1. Retinal extracts from each model were run with their respective controls to ensure accurate densitometric quantification. N=3 retinas/genotype. To highlight the differences in high-order complex, representative images have intentionally been saturated. However, all quantification was carried out using unsaturated images.

Experimental results confirmed the presence of abnormal complexes in Prph2^{Y141C/+}/Rho^{+/-} and Prph2^{K153Δ/+}/Rho^{+/-} retinas (FIG. 5, A). Quantification of the different complex forms was performed by measuring the signal intensity of each complex for PRPH2 and ROM1 and dividing it by the total signal intensity for the respective lane (FIG. 5, B and C). No changes in the distribution of PRPH2 complexes were observed in either Prph2^{Y141C/+}/Rho^{+/-} (FIG. 5(B), left) or Prph2^{K153Δ/+}/Rho^{+/-} (FIG. 5(B), right) compared to their respective heterozygous mutant counterparts. These findings indicate that the reduction of RHO does not affect the oligomerization of PRPH2.

### Reducing RHO Level Attenuates Retinal Gliosis in a Mutation-Independent Manner

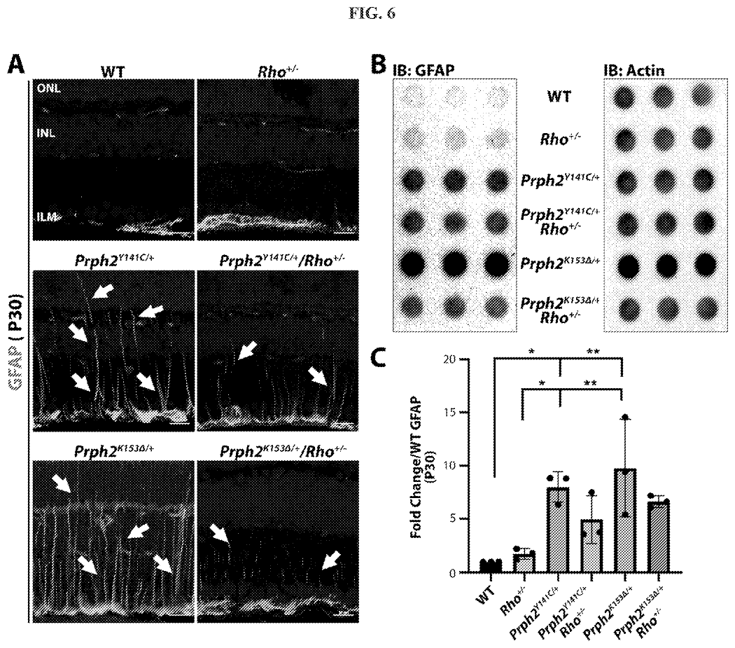

Müller cell gliosis, implicated in numerous retinal disorders, is characterized by the upregulation of glial fibrillary acidic protein (GFAP) as a non-specific marker of active gliosis and stress during retinal degeneration. Since reducing RHO levels in both Prph2 models led to functional and structural improvements, the impact of that reduction on GFAP upregulation and its cellular distribution was addressed. Immunofluorescence and immunoblotting were conducted on P30 Prph2^{Y141C/+} and Prph2^{K153Δ/+} retinas in the presence of WT levels of RHO or after ablation of one allele. Results show the reduction of RHO reduces gliosis in Prph2^{K153Δ/+}/Rho^{+/-} and Prph2^{Y141C/+}/Rho^{+/-} retinas. FIG. 6(A) shows representative immunostainings of P30 retinal sections from the indicated genotypes and stained for GFAP and DAPI. Arrows are used to mark GFAP infiltration across retinal layers in the models with wild-type RHO levels and their retractions on the Rho^{+/-} background. FIG. 6(B) shows representative immunodot blots probed for GFAP (left) and actin (right). FIG. 6(C) shows fold changes in GFAP relative to WT quantified from the immunodot blots presented in B and plotted as mean±SD. Samples from each genetic background were run with their own set of controls to allow for proper densitometric quantification. N=3-4 replicates/genotype. ** P<0.01, *** P<0.001, **** P<0.001 by one-way ANOVA (P=0.002) with Tukey's post-hoc comparison. Scale bar represents 20 μm.

In WT and Rho^{+/-} retinal sections, GFAP was predominantly localized in the nerve fiber layer consistent with previous literature (FIG. 6(A), upper panels), whereas in Prph2^{Y141C/+} and Prph2^{K153Δ/+}, GFAP extended to other retinal layers, including the ONL (FIG. 6(A), arrows in middle left and lower panels). Notably, the ablation of one Rho allele led to a reduction in retinal stress, evidenced by GFAP restriction to the nerve fiber, ganglion, and inner plexiform layers (FIG. 6(A), arrows in middle right and lower right panels). However, quantification by immunodot blotting of P30 retinal extracts from the listed genotypes (FIG. 6(B)) revealed a non-statistically significant trend in GFAP reduction upon elimination of one Rho complement (FIG. 6(C)). These findings collectively suggest that reducing RHO expression diminishes retinal stress associated with mutant Prph2 expression, although further research is warranted to confirm this observation and its significance.

Intravitreal Treatment with Rho-Specific ASO Slows Functional Decline in Prph2^{Y141C/+} Mice

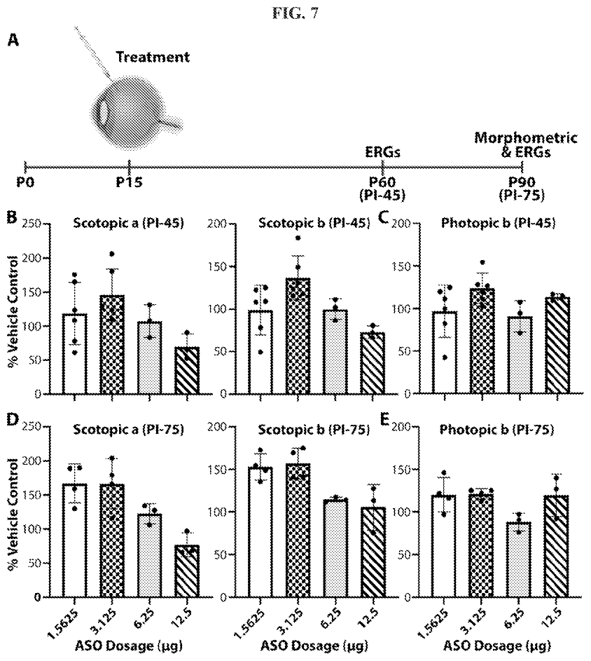

The potential of modulating RHO levels in a clinically relevant manner by the introduction of a rhodopsin specific ASO called mRho ASO1 was investigated. This ASO had already been tested and demonstrated efficacy in reducing endogenous mouse RHO. Murray et al showed “a dose-dependent reduction in rhodopsin mRNA was observed in eyes treated with mRho ASO1”. Titrations of a single intravitreal injection of mRho ASO1 were performed at ages P15 and P45 to explore the dose-dependent effects of early and late-stage therapeutic intervention in Prph2^{Y141C/+} mice. Therapeutic efficacy was assessed by recording ERGs at ages P60 and P90 to identify changes in visual function. Results were expressed as percentages normalized to the vehicle control values for each dosage. FIG. 7 relates to optimal mRho ASO1 dosage for early-stage therapeutic intervention and shows (A) design of the titration studies for the early-stage preclinical mRho ASO1 dosage, (B-C) scotopic a-, scotopic b-, and photopic b-wave amplitudes plotted as mean±SD of the percent of the independent vehicle control for early intervention dosage titrations measured at P60, and (D-E) scotopic a-, scotopic b-, and photopic b-wave amplitudes plotted as mean±SD of the percent of the independent vehicle control eye for early intervention dosage titrations measured at P90. N=3-6 eyes/treatment condition.

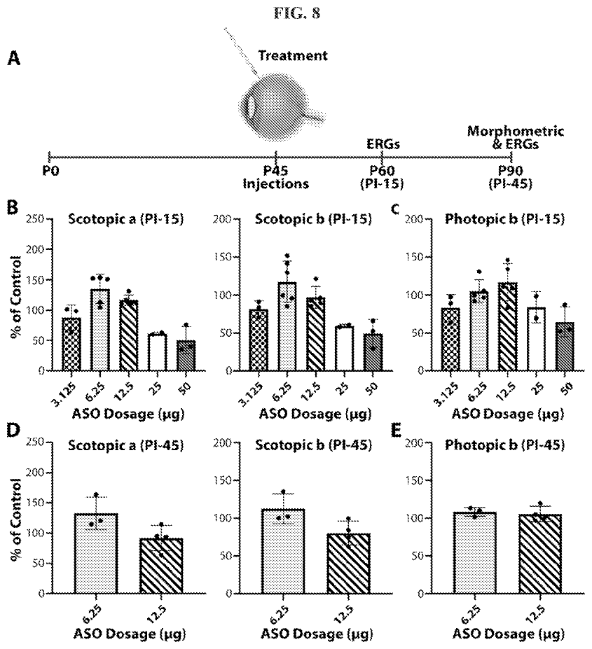

FIG. 8 relates to optimal mRho ASO1 dosage for late-stage intervention and shows (A) design of preclinical late-stage dosage titration studies, (B-C) scotopic a-, scotopic b-, and photopic b-wave amplitudes plotted as mean±SD of the percentage of the contralateral vehicle control for late intervention dosage titrations measured at P60, and (D-E) scotopic a-, scotopic b-, and photopic b-wave amplitudes plotted as mean±SD of the percentage of the contralateral vehicle control eye for late intervention dosage titrations measured at P90. N=3-6 eyes/treatment condition

Due to procedure related complications with the smaller eyes of P15 mice, the initial titration results are reported as the maximum amplitude (μV) measured for independent mRho ASO1 treated samples divided by the mean maximum amplitude of independent vehicle injected controls (FIG. 7(A)). The results for P45 injected mice are reported as the maximum amplitude measured for the ASO treated eye divided by that of the contralateral vehicle injected control eye (FIG. 8(A)).

The titration experiments revealed that optimal dosages were age at intervention dependent. Following P15 intervention, a dose of 3.125 μg showed the most improvement at P60 as determined by maximum scotopic a-(~146%), scotopic b-(~137%), and photopic b-(~124%) amplitudes compared to vehicle control eyes (FIG. 7, B and C). As the mice aged and the disease progressed, similar improvements were observed at P90 for both 1.56 μg (scotopic a-wave: ~ 167%, scotopic b-wave: 153%, and photopic b-wave: 120% ) and 3.125 μg (scotopic a-wave: ~ 167%, scotopic b-wave: ~ 157%, and photopic b-wave: ~ 121%) treated eyes compared to their vehicle control eyes (FIG. 7, D and E). Based on the observed increases in maximum amplitudes at P60 and P90 with the administration of 3.125 μg of mRho ASO1, we chose this dosage for the P15 intervention in the remainder of the study. As to the intervention at P45, improvements in maximum amplitudes were observed at P60 following the administration of 6.25 μg (scotopic a-wave: ~ 135%, scotopic b-wave: ~ 117%, and photopic b-wave: ~ 105%) and 12.5 μg (scotopic a-wave: ~ 117%, scotopic b-wave: ~ 97%, and photopic b-wave: ~ 117%) dosages (FIG. 8, B and C). The effects of the 6.25 μg dosage persisted at P90 (scotopic a-wave: ~ 133%, scotopic b-wave: ~ 112%, and photopic b-wave: ~ 108%), while the beneficial effects of the 12.5 μg dosage appeared to diminish (scotopic a-wave: ~ 92%, scotopic b-wave: ~ 80%, and photopic b-wave: ~ 105%).

To demonstrate the specificity of this ASO, a control ASO (5′-CCTATAGGACTATCCAGGAA-3′) (SEQ ID NO: 2) was generated and used. The impact of intravitreal injecting the control ASO was assessed to ensure there were no adverse effects on retinal function. For this study, the most effective dose of 6.25 μg in 1 μl was used to evaluate the efficacy of the control ASO in P30 Prph2^{Y141C/+} mice. At 15 days post injection (PI), mice were evaluated functionally, structurally and biochemically. Results were compared to P45 old untreated and PI-15 vehicle injected animals.

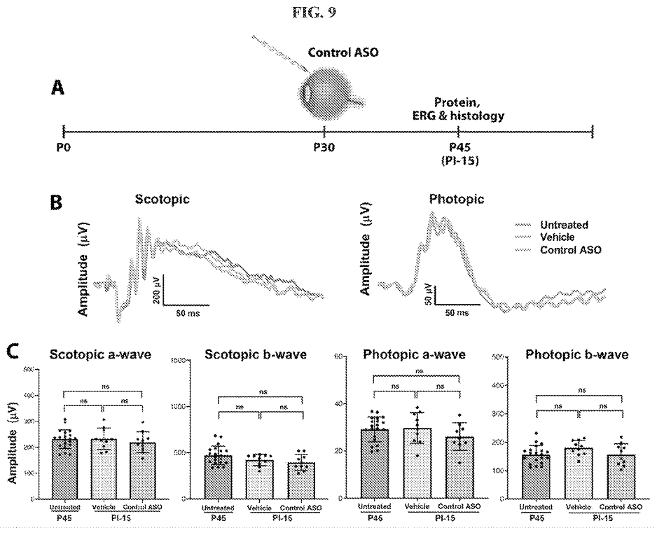

Control ASO does not alter the ERG responses of injected eyes. FIG. 9(A) shows schematic representation of the design for injection of the control ASO and the functional assessments. FIG. 9(B) shows representative waveforms of scotopic and photopic responses recorded 15 days post-injection of the control ASO. FIG. 9(C) shows mean±SD maximum amplitudes of scotopic a- and b-waves and photopic a- and b-waves of control ASO treated eyes relative to either untreated or vehicle treated eyes. ns: non-significant by one-way ANOVA test (N=20 for uninjected; 9-10 for vehicle or control ASO injected).

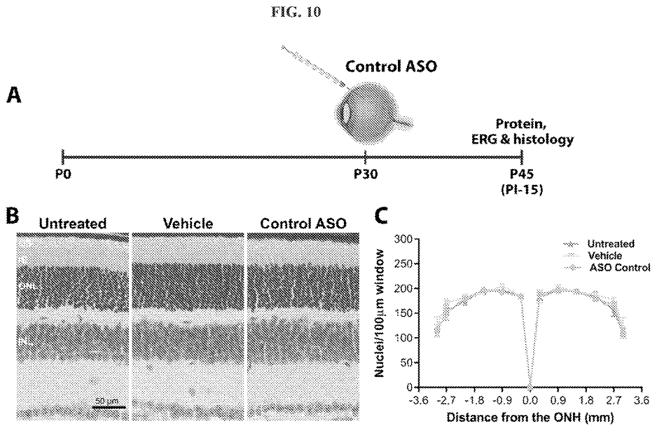

Control ASO does not alter the histologic appearance of the injected eyes. FIG. 10(A) shows schematic representation of the design for injection of the control ASO and the histologic evaluations. FIG. 10(B) shows representative light images of retinal cross sections from eyes injected with control ASO, compared to either untreated or vehicle-injected eyes, 15 days post-injection. FIG. 10(C) shows spidergram representing the count of photoreceptor nuclei in the outer nuclear layer of eyes injected with control ASO, compared to vehicle-injected and untreated eyes. (N=3). Statistical analysis by one-way ANOVA showed lack of significant differences between the groups.

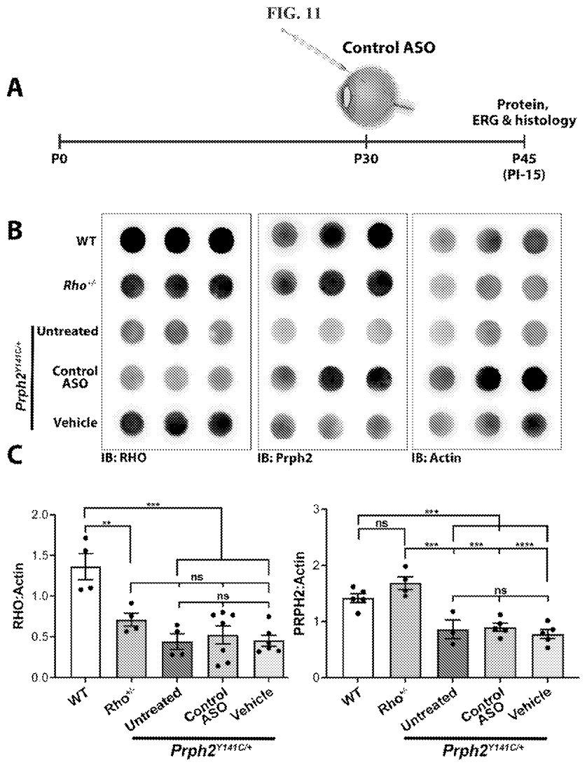

Control ASO does not alter the levels of RHO or PRPH2. FIG. 11(A) shows schematic representation of the design for injection of the control ASO and the immunoblot analyses. FIG. 11(B) shows representative immunodot blots used to assess the levels of RHO and PRPH2 in retinal extracts from eyes injected with control ASO, in comparison to untreated or vehicle-injected eyes, 15 days post-injection. Representative of three independent samples for each treatment are shown. FIG. 11(C) shows quantification of the levels of RHO (upper panel) or PRPH2 (lower panel) in retinal extracts from eyes injected with control ASO, in comparison to vehicle-injected or untreated eyes. (N=3 to 7) plotted as mean±SD. ns, non-significant; * P<0.05; *** P<0.001 by one-way ANOVA.

Functional assessments revealed scotopic a- and b-waves, as well as photopic a- and b-waves, were indistinguishable from the untreated or vehicle treated controls. Quantification of the ERG responses similarly showed no changes compared to the controls, both untreated and vehicle-injected. These results were further confirmed through light microscopy, and the number of photoreceptor nuclear counts showed no significant changes compared the control eyes. To verify that the injection of the control ASO did not affect the levels of endogenous RHO or PRPH2, immunodot blot analyses were conducted. Once again, no changes in the levels of RHO or PRPH2 were observed following the injection of the control ASO compared to untreated and treated samples.

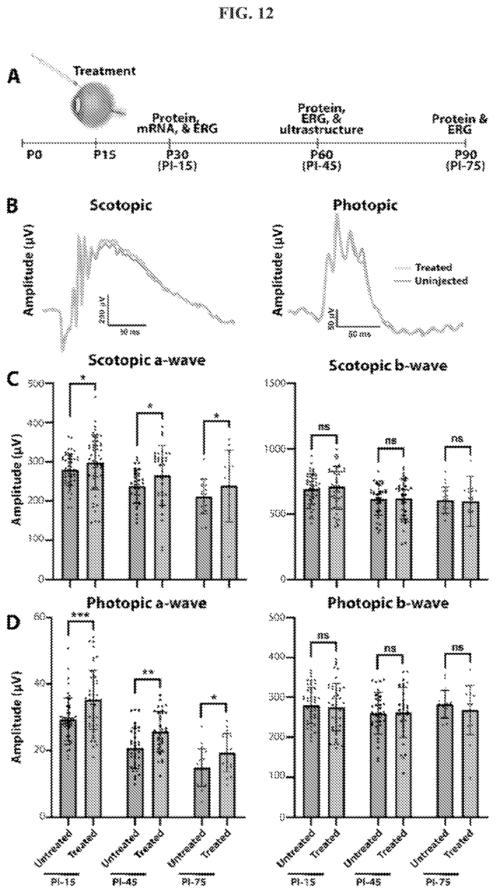

To further investigate the findings related to mRho ASO1 intervention at P15, ERGs were recorded from Prph2^{Y141C/+} mice at PI-15, PI-45 and P75, respectively. Results show that early-stage intervention with mRho ASO1 mitigates functional decline in Prph2^{Y141C/+} mice. FIG. 12 shows (A) Schematic representation of the design for early-stage preclinical intervention studies, (B) Representative waveforms of scotopic and photopic responses recorded 15 days after injection at P30, and mean±SD maximum amplitudes of (C) scotopic a-waves and scotopic b-wave and (D) photopic a-wave and b-wave amplitudes of treated (3.125 μg mRho ASO1) and untreated contralateral control eyes recorded in preclinical early injection studies at ages P30, P60, and P90. * P<0.05, ** P<0.01, *** P<0.001 by Mann-Whitney U test (PI-15: N=51, PI-45: N=34, and PI-75: N=18). Comparing ERG values for ASO-treated eyes with their untreated contralateral eyes revealed significant increases in mean treated maximal scotopic a-wave amplitudes at each evaluated time point (PI-15: ~ 107%, PI-45: ~ 112%, and PI-75: ~ 113%), while b-wave maximum amplitudes remained unchanged following therapeutic intervention (FIG. 12, B and C). This pattern of specific improvement was also observed in photopic responses, where photopic a-wave maximum amplitudes were significantly improved at each time point of assessment compared to their corresponding control eye (PI-15: ~ 120%, PI-45: ~ 124% , and PI-75: ~ 130%), and no significant differences were observed when evaluating photopic b-wave maximum amplitudes (FIG. 12D).

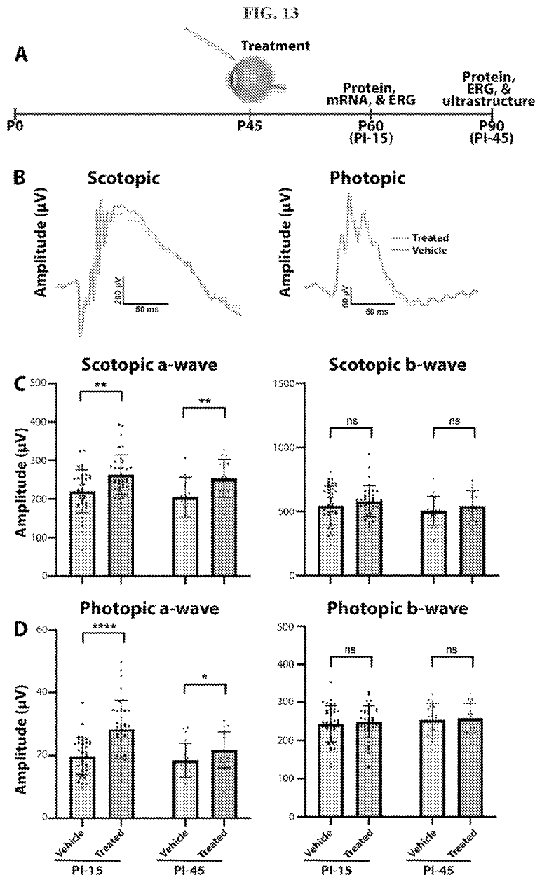

Results also show that late-stage intervention with mRho ASO1 preserves functional performance in Prph2^{Y141C/+} mice. Functional changes were evaluated at PI-15 and PI-45 following P45 mRho ASO1 intervention. FIG. 13 shows (A) Design of late-stage preclinical intervention studies, (B) Representative scotopic and photopic waveforms recorded at 15 and 45 days post injections at P45, and (C-D) Mean maximum amplitudes of scotopic a-waves and b-waves, as well as photopic a-waves and b-waves were plotted as mean±SD for treated eyes (6.25 μg mRho ASO1) and vehicle control contralateral eyes in the late-stage intervention studies conducted at P60 and P90. * P<0.05, ** P<0.01, *** P<0.001, **** P<0.0001 by Mann-Whitney U test (PI-45: N=38 and PI-75: N=18). Comparing ASO-treated eyes with their vehicle control contralateral counterparts at PI-15 revealed higher scotopic (~120%) and photopic (~144%) a-wave maximum amplitudes, while no observable differences were noted in scotopic and photopic b-wave maximum amplitudes (FIG. 13, B-D). This improvement in a-wave amplitudes persisted at PI-45 (scotopic a-wave: ~124% and photopic a-wave: ~118%), with no enhancement observed in b-wave maximum amplitudes (FIG. 13, C and D).

### ASO Treatment Lowers the Ratio of RHO to PRPH2

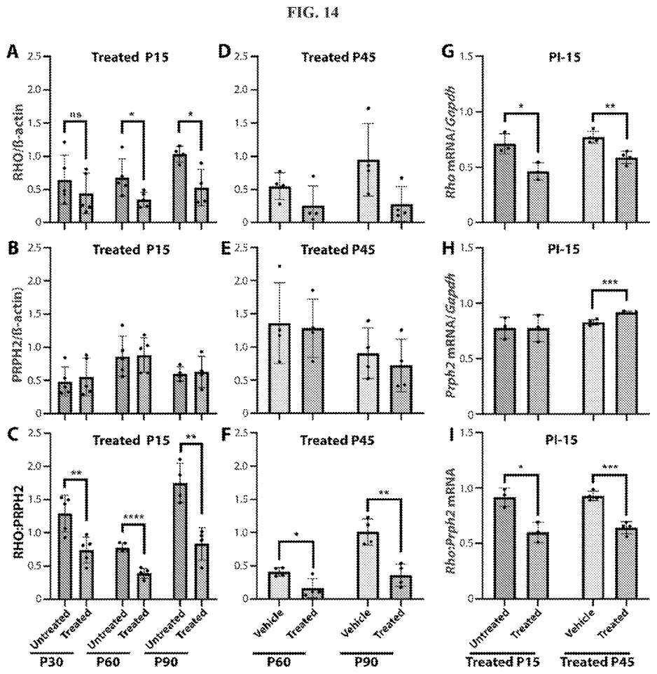

Next, the effects of ASO intervention on RHO and PRPH2 levels were explored by quantifying protein amounts through immunodot blots and employing a two-tailed t-test for statistical analysis. Results showed that treatment with mRho ASO1 effectively reduced protein and transcript levels in Prph2^{Y141C/+} mice. The evaluation time points following intravitreal injection at P15 included ages P30, P60, and P90 corresponding to PI-15, PI-45, and PI-75. FIG. 14 shows quantification of (A) RHO and (B) PRPH2 in retinal extracts at P30 (PI-15), P60 (PI-45), and P90 (PI-75) after injections with mRho ASO1 at P15 (3.125 μg mRho ASO1), (C) Graphs depicting the ratio of RHO to PRPH2 following P15 injection, determined by dividing the RHO signal intensity value by that of PRPH2, Quantification of (D) RHO and (E) PRPH2 in retinal extracts at P60 (PI-15) and P90 (PI-45) after injections with mRho ASO1 at P45 (6.25 μg mRho ASO1), (F) Graphs illustrating the ratio of RHO to PRPH2 following P45 injections, Transcript levels assessed by qRT-PCR at PI-15, with quantification of (G) Rho and (H) Prph2 mRNA transcript levels relative to Gapdh for P15 and P45 injected samples, and (I) Quantification of Rho mRNA transcript levels relative to Prph2 for P15 and P45 injected samples. Data are presented as mean±SD. * P<0.05, ** P<0.01, *** P<0.001, **** P<0.0001 as determined by student t-test analysis. N=3-5 retinas per treatment condition.

Treated eyes exhibited a reduction in mean RHO levels by ~32% at P30, and ~49% at P60, and P90, albeit this reduction was statistically insignificant at P30 (FIG. 14A). ASO administration at P15 did not significantly affect PRPH2 protein levels at any of the assessed time points (FIG. 14B). A statistically significant reduction in mean ratio of RHO to PRPH2 was observed at all three time points (P30: ~ 43%, P60: ~ 49%, and P90: 52%) (FIG. 14C). These improved ratios indicate the successful achievement of the desired therapeutic effect by efficiently reducing RHO levels. It is important to avoid excessive reduction of RHO, as it may exacerbate the degeneration and subsequently reduce PRPH2 levels.

Protein quantification was performed at PI-15 and PI-45 for intervention at P45 (P60 and P90, respectively). Average RHO protein levels showed a reduction of ~53% at P60 and ~71% at P90 compared to contralateral control eyes (FIG. 14D). Similar to the early intervention group, ASO treatment did not induce significant changes in average PRPH2 levels at both P60 and P90, although a slight decrease in the mean was observed at P90 (FIG. 14E). However, assessing the ration of RHO to PRPH2 revealed that the therapeutic effect of ASO treatment was successfully achieved, resulting in a decreased mean ratio of RHO to PRPH2 (P60: ~ 60% and P90: ~ 64%). Furthermore, this reduction was statistically significant at both time points (FIG. 14F).

To verify the mechanism of action for mRho ASO1 intervention, qRT-PCR was employed to quantify Rho and Prph2 transcript levels post injection. Table 2 below shows the primers used.

TABLE 2
qRT-PCR Primers

| Gene of | | |
|---|---|---|
| Interest | Sequence (5′-3′) | FWD/REV |
| Rho | CACTCCATGGCTACTTCGTCTTT (SEQ ID NO: 3) | FWD |
| | TGGCCCAAATGTTGCTGGATAGTTTTT (SEQ ID NO: 4) | REV |
| Prph2 | GGAGGTCAAAGATCGCATCA (SEQ ID NO: 5) | FWD |
| | GCTCCTCAGTCTGATGGTCATA (SEQ ID NO: 6) | REV |
| Gapdh | GAAGGTCGGTGTGAACGG (SEQ ID NO: 7) | FWD |
| | ATGAAGGGGTCGTTGATGGC (SEQ ID NO: 8) | REV |

Quantification of Rho transcript levels normalized to that of Gapdh revealed significant decreases in mRNA levels following injections at P15 (~35%) and P45 (~24%) (FIG. 14G). In contrast, evaluation of Prph2 transcript levels normalized to Gapdh revealed no significant changes between uninjected and treated eyes following P15 injections. However, a significant increase (~11%) in Prph2 transcript levels was observed following P45 injection of mRho ASO1 compared to vehicle control (FIG. 14H). The relative abundance of the Rho transcript was assessed compared to Prph2 transcript to gain insight into how the intervention affects this ratio. This revealed similar levels of significant reduction in the ratio of Rho to Prph2 mRNA following P15 (~24%) and P45 (~31%) injections (FIG. 14I). These findings further support the mechanism of action of mRho ASO1 intervention in effectively reducing Rho transcript levels while exhibiting no significant impact on Prph2 transcript levels.

Age-Dependent Impact of mRho ASO1 Intervention in Mitigating Histopathologic Defects

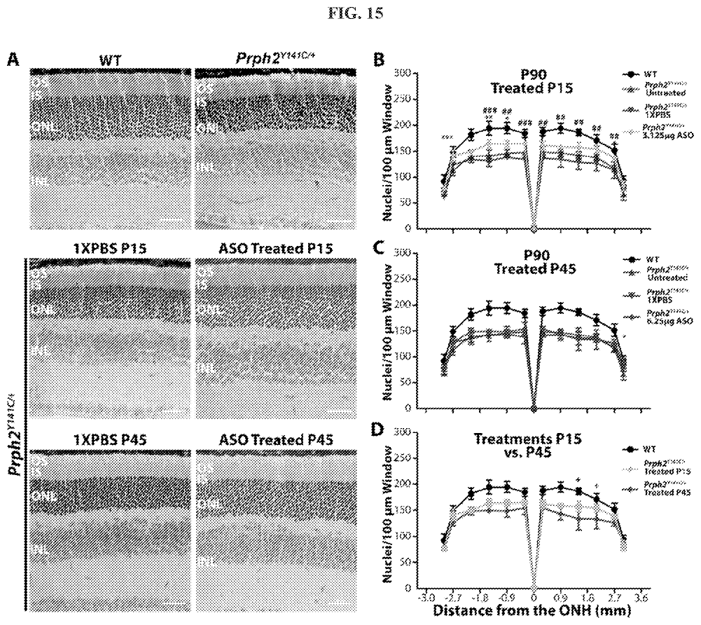

To assess the histologic changes in Prph2^{Y141C/+} following ASO treatment, morphometric analyses were conducted on retinal sections from P90 mice injected with either 3.125 μg mRho ASO1 at P15 or 6.25 μg mRho ASO1 at P45. Results showed early-stage treatment with mRho ASO1 successfully delays ONL thinning in Prph2^{Y141C/+} mice. FIG. 15 shows (A) Representative images of retinal sections stained with H&E at P90, (B-D) Nuclear counts from 100 μm-windows at every 500 μm distance from the optic nerve and across the superior-inferior plane of retinal sections collected from P90 un-injected, 1X PBS injected as a control and mRho ASO1 injected animals following (B) early (P15) or (C) late-stage (P45) therapeutic intervention. (B-C) WT and Prph2^{Y141C/+} controls were added for comparison. For FIG. 15(B-C) *P<0.05, ** P<0.01, *** P<0.001 by two-way ANOVA with Tukey's post-hoc comparison. In FIG. 15, *denotes comparisons between Prph2^{Y141C/+} and (B) Prph2^{Y141C/+} 3.125 μg ASO or (C) Prph2^{Y141C/+}6.25 μg ASO. #denotes comparisons between (B) Prph2^{Y141C/+} 3.125 μg ASO and P15 injected Prph2^{Y141C/+} 1X PBS. In FIG. 15(D) plotted are mean±SD values from (B) and (C) for WT, Prph2^{Y141C/+} P15 Injected 3.125 μg ASO, and P45 injected 6.25 μg ASO Prph2^{Y141C/+} for ease of comparison. In. FIG. 15, + denotes comparisons P15 injected and P45 injected. N=3 animals for all genotypes and experimental conditions. Scale bar corresponds to 50 μm.

Animals injected at P15 exhibited significant improvement in the number of photoreceptor nuclei compared to vehicle and untreated controls (FIG. 15, A and B). However, injections at P45 did not show a significant improvement in number of photoreceptor nuclei, except for one area in the far superior periphery (FIG. 15C). When directly comparing the two intervention time points, it is evident that P15 intervention leads to improved ONL nuclear count throughout most of the retina, with a statistically significant improvement in the superior portion of the retina (FIG. 15D). Taken together, these results demonstrate the efficacy of early intervention using mRho ASO1 as a therapeutic strategy to delay photoreceptor death caused by the Prph2^{Y141C/+} pathogenic variant.

mRho ASO1 Intervention Improves OS Ultrastructure

Ultrastructural examination of ROSs was performed 45 days post injection at both P15 and P45. ASO treated photoreceptors showed notable improvements in their upright appearance, organization and a reduction in whorl formations, which are characteristic features of mutant PRPH2 murine retinas.

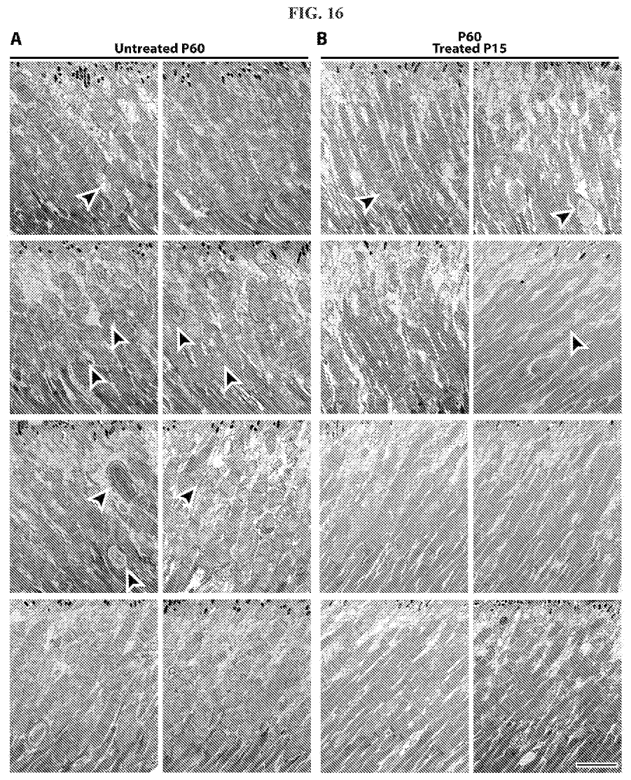

mRho ASO1 intervention at P15 leads to enhanced OS ultrastructure and reduced formation of whorl like structures. FIG. 16 shows representative TEM images captured from retinas at P60 showing untreated contralateral control (A) and 3.125 μg mRho ASO1 injected eyes at P15 and evaluated at P60 (PI-45) (B). Scale bar, 6 μm. Images are from one animal to illustrate improvements observed throughout the retina.

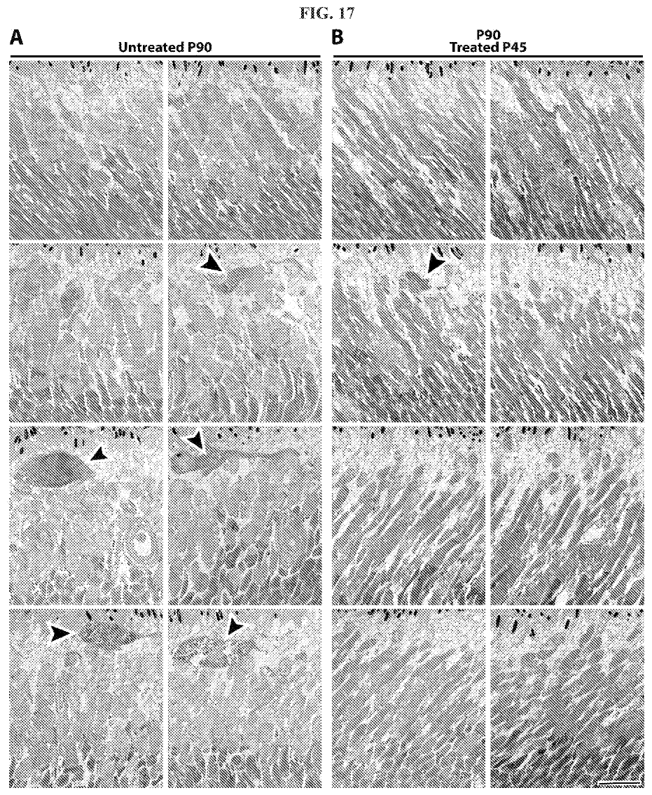

mRho ASO1 intervention at P45 leads to enhanced OS ultrastructure, reduced formation of whorl like structures, and decreased infiltration of mononuclear cells. FIG. 17 shows representative TEM images of retinas at P90 showing untreated contralateral eyes (A) and 6.25 μg mRho ASO1 treated eyes at P45 and evaluated at P90 (PI-45) (B). Scale bar, 6 μm. Images are from one animal to illustrate the widespread improvements observed throughout the retina. Arrowheads indicate the observed mononuclear cell infiltration in the subretinal space.

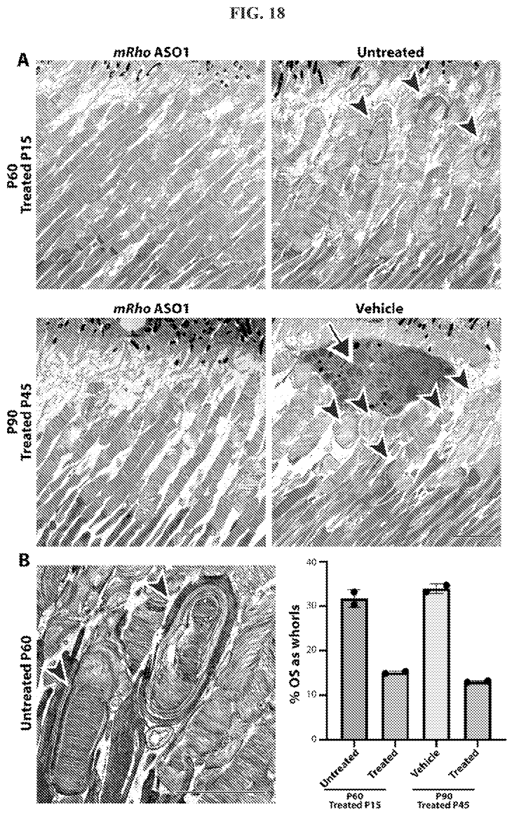

mRho ASO1 treatment leads to improvements in ROS ultrastructure and reduced immune cell infiltration. FIG. 18 shows (A) representative low-magnification TEM images of tannic acid/uranyl acetate-stained retinas from mRho ASO1 treated eyes 45 days following treatment at P15 (3.125 μg mRho ASO1) and P45 (6.25 μg mRho ASO1) and untreated contralateral control eyes, and (B) a representative image of a whorl-like structure present in P60 uninjected contralateral Prph2^{Y141C/+} eye (left) and quantification of whorls presented as a percentage of the total number of counted OSs. N=144-307 OSs counted per retina (right). N=2 retinas per treatment condition. An arrow indicates mononuclear cell infiltration while arrowheads highlight whorl-like structures. Scale bar, 5 μm. Error bars represent mean±SD.

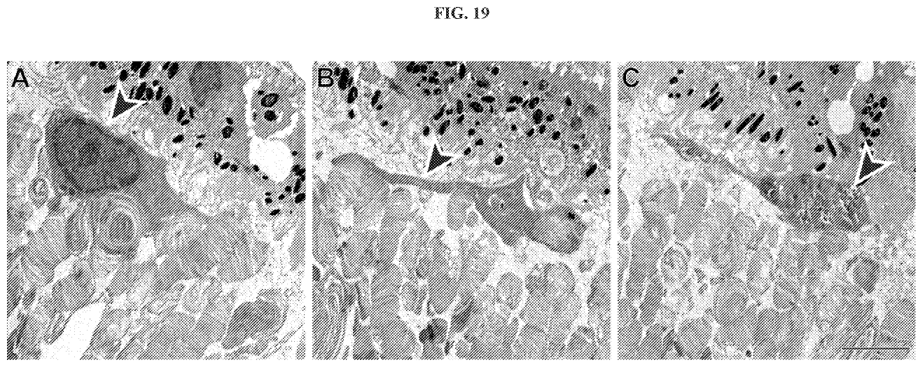

Evaluating the percentage these whorls represent of the total ROSs throughout the retina revealed ~50% reduction after early ASO injections and ~62% reduction associated with late therapeutic intervention compared to contralateral controls. Additionally, numerous nucleated cells were frequently observed in the subretinal space of control P90 Prph2^{Y141C/+} eyes, containing a large amount of phagocytosed material. While these cells were also present in ASO-injected animals, their presence was significantly reduced. Evaluation of PBS-treated control Prph2^{Y141C/+} eyes at P45 and collected at P90 revealed the presence of likely activated microglia. FIG. 19 relates to defining characteristics of presumed microglial immune cell infiltration and shows representative TEM images of tannic acid/uranyl acetate-stained retinas from Prph2^{Y141C/+} eyes with control injection of PBS at P45 and collected at P90, (A) Mononucleated cell located in the subretinal space, (B) Extended processes observed as a characteristic of these cells, (C) Cells found to possess a large amount of phagocytosed material. Arrowheads highlight: (A) nuclei, (B) extended processes, and (C) phagocytosed material. Scale bar, 5 μm. These cells were characterized by their nucleated appearance (FIG. 19(A)), localization next to the RPE, extended processes (FIG. 19(B)), and the presence of phagocytized material and numerous lysosomes (FIG. 19(C), arrowhead).

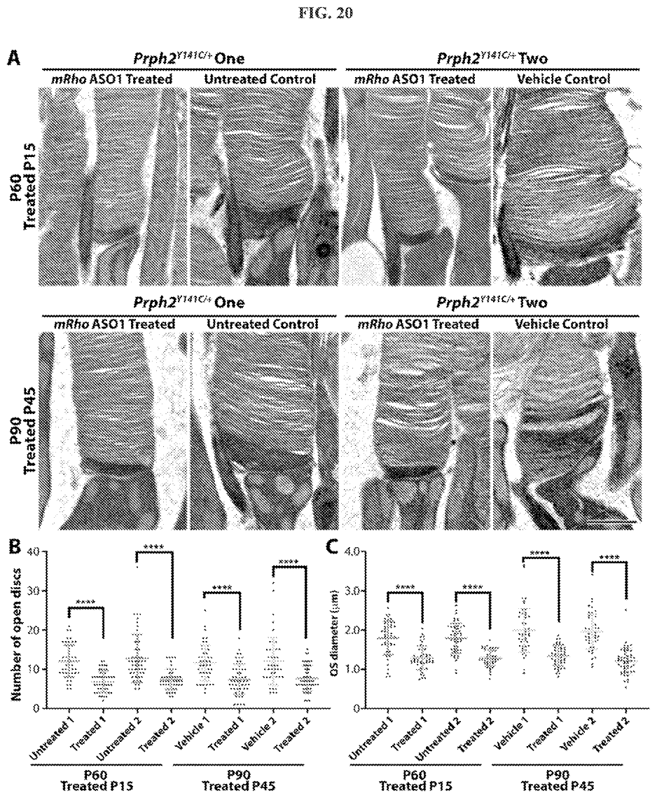

To further investigate the observed improvements in ROS ultrastructural, measurements of OS diameters and open disc counts were performed at PI-45 following both early and late treatment to quantitatively assess the known morphological defects. Injection of mRho ASO1 led to significant improvements in the morphogenesis and ultrastructure of OSs in Prph2^{Y141C/+} mice. FIG. 20 shows (A) Representative high-magnification TEM images of tannic acid/uranyl acetate-stained retinas taken from P90 mRho ASO1 treated and contralateral control eyes that were injected at P15 (3.125 μg mRho ASO1) and P45 (6.25 μg mRho ASO1), and (B-C) Quantification of open discs at the base of the OS (B) and OS diameters (C) in mRho ASO1 injected and contralateral control eyes 45 days after treatment. Each data point represents a single OS. * P<0.05, ** P<0.01, *** P<0.001, **** P<0.0001 by one-way ANOVA (P<0.0001 for both open discs and OS diameter) with Tukey's post-hoc comparison. N=50 OSs per each sample for B and C. Scale bar, 1 μm. Error bars represent mean±SD.

Table 3 shows quantification of open discs and OS diameter for P15 and PI-45 Prph2^{Y141C/+} mRho ASO treated and their corresponding untreated contralateral control eyes.

TABLE 3

| | | |
|---|---|---|
| | Number of | OS Diameter |
| | Open Discs | (μm) |
| Samples | (mean ± SD) | (mean ± SD) |

P15 Prph2^{Y141C/+} injected mice with mRho ASO1

| | | |
|---|---|---|
| P15 Contralateral Control One | 12.0 ± 0.6 | 1.80 ± 0.06 |
| P15 Treated Sample One | 6.8 ± 0.4 | 1.29 ± 0.04 |
| P15 Contralateral Control Two | 12.8 ± 0.9 | 1.79 ± 0.05 |
| P15 Treated Sample Two | 7.4 ± 0.4 | 1.29 ± 0.03 |

P45 Prph2^{Y141C/+} injected mice with mRho ASO1

| | | |
|---|---|---|
| P45 Contralateral Control One | 11.7 ± 0.6 | 2.00 ± 0.08 |
| P45 Treated Sample One | 7.4 ± 0.5 | 1.35 ± 0.04 |
| P45 Contralateral Control Two | 12.0 ± 0.9 | 1.97 ± 0.06 |
| P45 Treated Sample Two | 7.7 ± 0.5 | 1.21 ± 0.05 |

Treatment with mRho ASO1 resulted in a reduction in the number of open nascent discs compared to contralateral controls following both time points of intervention (P15 intervention: control ~12.4 and treated 7.1) (P45 intervention: control ~11.8 and treated ~7.6). This decrease was also evident in OS diameter measurements (P15 intervention: control ~1.8 μm and treated ~1.3 μm) (P45 intervention: control ~2 μm and treated ~1.3 μm) (FIG. 20C and Table 1). Similar to what was observed with genetic ablation, these results indicate that mitigation of these morphological defects plays a crucial role in driving the observed improvements in ROS ultrastructural following mRho ASO1 administration.

What is claimed is:

1. A method for the treatment of peripherin-2 (PRPH2) associated diseases in a subject, comprising:
administering a composition comprising a therapeutically effective amount of an inhibitor of the production of rhodopsin to the subject.

2. The method of claim 1, wherein the inhibitor of the production of rhodopsin is an antisense oligonucleotide.

3. The method of claim 1, wherein the inhibitor of the production of rhodopsin is an antisense oligonucleotide having SEQ ID NO:1.

4. The method of claim 1, wherein the composition further comprises a pharmaceutically acceptable excipient, adjuvant, carrier, buffer, stabilizer, or mixture thereof.

5. The method of claim 1, wherein the composition is administered by intravitreal injection to the subject.

6. The method of claim 1, wherein administering the composition results in a decrease in the ratio of rhodopsin to peripherin-2 in retinas of the subject.

7. A composition for the prevention or treatment of peripherin-2 (PRPH2) related diseases, comprising a therapeutically effective amount of an inhibitor of the production of rhodopsin.

8. The composition of claim 7, wherein the inhibitor of the production of rhodopsin is an antisense oligonucleotide.

9. The composition of claim 7, wherein the inhibitor of the production of rhodopsin is an antisense oligonucleotide having SEQ ID NO:1.

10. The composition of claim 7, further comprising a pharmaceutically acceptable excipient, adjuvant, carrier, buffer, stabilizer, or mixture thereof.


SEQUENCE LISTING (8 sequences)

SEQ ID NO: 1 - DNA, 20 bp, synthetic construct
  source 1..20: mol_type = other DNA
agctactatgtgttccattc

SEQ ID NO: 2 - DNA, 20 bp, synthetic construct
  source 1..20: mol_type = other DNA
cctataggactatccaggaa

SEQ ID NO: 3 - RNA, 23 nt, synthetic construct
  source 1..23: mol_type = other RNA
cactccatggctacttcgtcttt

SEQ ID NO: 4 - RNA, 27 nt, synthetic construct
  source 1..27: mol_type = other RNA
tggcccaaatgttgctggatagttttt

SEQ ID NO: 5 - RNA, 20 nt, synthetic construct
  source 1..20: mol_type = other RNA
ggaggtcaaagatcgcatca

SEQ ID NO: 6 - RNA, 22 nt, synthetic construct
  source 1..22: mol_type = other RNA
gctcctcagtctgatggtcata

SEQ ID NO: 7 - RNA, 18 nt, synthetic construct
  source 1..18: mol_type = other RNA
gaaggtcggtgtgaacgg

SEQ ID NO: 8 - RNA, 20 nt, synthetic construct
  source 1..20: mol_type = other RNA
atgaaggggtcgttgatggc